(19) United States
(12) Patent Application Publication
(10) Pub. No.: US 2026/0049114 A1
(43) Pub. Date: Feb. 19, 2026

(54) FUSION PROTEIN FOR THE PREVENTION, TREATMENT OR AMELIORATION OF KIDNEY DISEASES

(71) Applicants: UCB BIOPHARMA SRL, Brussels (BE); FIVE PRIME THERAPEUTICS, INC., Thousand Oaks, CA (US)

(72) Inventors: Lara Kevorkian, Slough (GB); Timothy Scott Johnson, Slough (GB); Breda Twomey, Slough (GB); Janine Powers, Thousand Oaks, CA (US); Danielle E. Kellar, Thousand Oaks, CA (US)

(21) Appl. No.: 18/875,022

(22) PCT Filed: Jun. 14, 2023
(86) PCT No.: PCT/EP2023/065967

§ 371 Date: Dec. 13, 2024

(30) Foreign Application Priority Data
Jun. 15, 2022 (WO) PCT/EP2022/066348

(51) Int. Cl. C07K 14/495; A61K 38/00; A61P 13/12; C07K 16/18

(52) U.S. Cl. CPC C07K 14/495; A61P 13/12; C07K 16/18; A61K 38/00; C07K 2317/55; C07K 2317/565; C07K 2319/00

(57) ABSTRACT

The invention relates to proteins for use in the prevention, treatment or amelioration of kidney diseases or kidney damage, more particularly where the protein is a follistatin-fusion protein.

### CROSS REFERENCE TO RELATED APPLICATIONS

This application is a 35 U.S.C. § 371 national phase entry of International Application No. PCT/EP2023/065967, filed Jun. 14, 2023, which claims priority to European Patent Application No. PCT/EP2022/066348, filed Jun. 15, 2022, the disclosures of which are hereby incorporated by reference in their entireties.

### INCORPORATION BY REFERENCE OF MATERIAL SUBMITTED ELECTRONICALLY

The application is being with a sequence listing in electronic format. The sequence listing is provided as a file entitled “70815_SubSeqListing.xml”, created Dec. 27, 2024, which is 49,488 bytes in size. The subject matter of the sequence listing is incorporated by reference herein in its entirety.

### FIELD OF THE INVENTION

The invention relates to proteins for use in the prevention, treatment or amelioration of kidney diseases or kidney damage, more particularly where the protein is a follistatin-fusion protein.

### BACKGROUND OF THE INVENTION

Chronic kidney disease (CKD) affects approximately 10-13% of the aging populations across the world and is associated with significant mortality and morbidity. It is estimated that 1.5-2% of those with CKD will progress to end stage kidney disease (ESKD) and the two leading causes of ESKD are diabetes mellitus and hypertension which despite medical management are still progressive. For the past 20 years treatment options for CKD have relied on control of hypertension and blood glucose but are grossly inadequate in the long-term disease management. Treatment of ESKD relies on dialysis and kidney transplant neither of which have great favourable outcomes for those patients with the progressive form of disease. New therapies are desperately needed and especially those that incorporate novel mechanisms (Yahr et al (2022); Ammirati et al (2020)).

A common feature of failing kidneys is the build-up of fibrotic areas within the tissue which builds up gradually over time and ultimately can lead to progressive failure of the normal functioning of the organ. Fibrotic diseases are diseases which are characterised by an aberrant wound healing response in which excess fibrous connective tissue is formed in an organ or tissue. The deposition and accumulation of excess extracellular matrix components such as collagen and fibronectin results in the hardening and scarring of tissues, causing a pathological remodelling of the organ and can ultimately lead to organ failure (Wynn & Ramalingam (2012)).

Follistatin is an autocrine glycoprotein which has as its primary function the binding and neutralisation of members of the TGF-beta superfamily and in particular activin. It is known to exist in several different forms, including a 315-amino acid polypeptide (designated FST315); and a 288-amino acid polypeptide (designated FST288). Both FST315 and FST288 have high affinity for activin (activin A & activin B) as well as for myostatin. In particular, follistatin can bind to and inhibit myostatin, which is a negative regulator of skeletal muscle mass. Follistatin is a protein of interest that could be used, as such or as a fusion moiety for a fusion protein, in therapy in a number of conditions, including muscle disorders such as muscular dystrophy (WO2015/187977 and WO2017/152090), or yet diseases or disorders such as in inflammatory bowel disease, ulcerative colitis and Crohn's Disease (WO03/006057).

However, there remains a need to identify effective therapies for use in treatment and prevention of kidney diseases or kidney damage.

### SUMMARY OF THE INVENTION

In a first aspect, the invention provides a follistatin-fusion protein for use in the treatment, amelioration or prevention of kidney diseases, such as chronic kidney diseases (CKD), or kidney damage, wherein the follistatin-fusion protein comprises: a. a follistatin moiety, b. an antibody moiety, and optionally c. a linker between the follistatin moiety and the antibody moiety.

In a second aspect, the invention relates to a method of treating a patient in need of therapy for the treatment, amelioration or prevention of kidney diseases, such as chronic kidney diseases (CKD), or kidney damage, the method comprising administering a therapeutically effective amount of a follistatin-fusion protein, wherein the follistatin-fusion protein comprises: a. a follistatin moiety, b. an antibody moiety, and optionally c. a linker between the follistatin moiety and the antibody moiety.

In a third aspect, the invention describes the use of a follistatin-fusion protein in the manufacture of a medicament for the treatment, amelioration or prevention of kidney diseases, such as chronic kidney diseases (CKD), or kidney damage, wherein the follistatin-fusion protein comprises: a. a follistatin moiety, b. an antibody moiety, and optionally c. a linker between the follistatin moiety and the antibody moiety.

In particular, 1) the kidney disease is preferably a chronic kidney disease (CKD) such as primary glomerulonephritis, secondary glomerulonephritis, diabetic nephropathy, hypertensive nephrosclerosis, focal segmental glomerulosclerosis (FSGS), IgA nephropathy (IgAN), Mesangial proliferative glomerulonephritis, Membranous Nephropathy (MN), Minimal Change Disease (MCD), polycystic kidney disease, chronic allograft nephropathy, CKD-associated mineral and bone disorder (CKD-MBD) and Goodpastures disease and 2) the kidney damage is a) kidney fibrosis, or b) is associated with other diseases such as diabetes mellitus, hypertension, cardiovascular disease or bone disorders. Said kidney diseases or kidney damage can be the result of a therapeutic treatment, such as a treatment with chemotherapy.

### BRIEF DESCRIPTION OF THE FIGURES

FIG. 1: Schematic drawing showing the FST288 and FST315 molecules aligned with a bound activin molecule.

FIG. 2: Follistatin modulation of Adriamycin-driven chronic kidney disease model study in mouse with delivery of therapeutic protein using hydrodynamic transfection. (FIG. 2A) Hydroxyproline kidney content was determined at study termination as a surrogate for collagen accumulation and fibrosis of the kidney, without disease=Naïve, with disease and saline treatment=Saline, and with disease and follistatin treatment=Follistatin. (FIG. 2B) Serum creatinine was measured at study termination as a surrogate for kidney function, without disease=Naïve, with disease and saline treatment=Saline, and with disease and follistatin treatment=Follistatin. (FIG. 2C) Conditional survival showing status of animals for duration of the study and at study termination, where all the living animals that survived disease were terminated in either the saline- or follistatin-treated disease groups. (FIG. 2D) Picrosirius Red (PSR) staining as a surrogate for Collagen content of selected kidneys showing 1 kidney from the Naïve group, 3 separate kidneys from the Saline-treated disease group and 3 separate kidneys from the Follistatin-treated disease group. (FIG. 2E) All the kidneys from the study were stained with PSR and analysed to determine the relative Percentage Area of Collagen in the 3 different groups where the mice without disease=Naïve, with disease and saline-treated=Saline and with disease and follistatin-treated=Follistatin; Statistics were performed comparing the saline, and follistatin-treated groups using the ordinary one-way ANOVA with Tukey's multiple comparison test and **** represent p<0.0001. Group sizes, Naïve n=8, Saline n=21, Follistatin n=18.

FIG. 3: Comparison of different follistatin moieties for modulation of unilateral ureter obstruction model (UUO) of acute kidney disease in mouse with delivery of the therapeutic proteins by hydrodynamic transfection using standard methods and commercial plasmids. (FIG. 3A) Picrosirius Red (PSR) staining as a surrogate for Collagen content of selected kidneys showing 1 kidney without disease (non-operated)=Non-Op, and 4 kidneys from the UUO disease group treated with a control plasmid, secreted alkaline phosphatase=SEAP control, a plasmid containing FST315(HBM)-Fab and a plasmid containing FST315-Fab. (FIG. 3B) All the kidneys from the study were stained with PSR and analysed to determine the relative Percentage Area of Collagen in the different groups described in FIG. 3A; Statistics were performed using the ordinary one-way ANOVA with Tukey's multiple comparison test with NS denoting non-significance, * p<0.05. (FIG. 3C) Changes in body weight of mice in the different groups; control plasmid (SEAP), FST315(HBM)-Fab or FST315-Fab plasmids. Group sizes, Non-operated n=4, Seap control n=8, FST315(HBM)-Fab n=7, FST315-Fab n=7

FIG. 4: Comparison of follistatin protein moieties, FST315-Fab and FST315(HBM)-Fab, with the latter as a dose-response, for modulation in the unilateral ureter obstruction model (UUO) of acute kidney disease in mouse with delivery of the therapeutic proteins directly using sub-cutaneous administration. (FIG. 4A) Morphological representation of whole kidneys at low magnification stained with PSR from the UUO mouse study showing a kidney without disease or non-operated=Normal in the left panel, and 2 UUO kidneys, the middle panel is a UUO diseased kidney with vehicle treatment=21 Day UUO, and the right panel a UUO kidney treated with FST315(HBM)-Fab at 10 mg/kg/day. The associated histological scores are also shown alongside. (FIG. 4B) Morphological representation of a kidney segment at high magnification stained with PSR showing a UUO diseased kidney with vehicle treatment (top panel) and a UUO diseased kidney with 10 mg/kg FST315(HBM)-Fab treatment (bottom panel). (FIG. 4C) Morphological representation of a kidney segment at medium magnification stained with PSR showing a representative normal kidney (Non-op) and UUO diseased representative kidneys treated with vehicle or FST315-Fab at 2 mg/kg, FST315(HBM)-Fab at 10, 2, 0.2 and 0.02 mg/kg. (FIG. 4D) All the kidneys from the study were stained with PSR and scanned to determine the relative Percentage Area of Collagen in the different groups which were non-operated or healthy kidneys=Non Op (n=4), UUO diseased kidneys treated with Vehicle (n=8), 2 mg/kg 315FST-Fab (n=8), 10 mg/kg 315FST(HBM)-Fab (n=6), 2 mg/kg 315FST(HBM)-Fab (n=8), 0.2 mg/kg 315FST(HBM)-Fab (n=8) and 0.02 mg/kg 315FST(HBM)-Fab (n=8); Statistics were performed using ordinary one-way ANOVA with uncorrected Fisher's LSD multiple comparison tests where NS denotes non-significance, * p<0.05, and **** p<0.0001. (FIG. 4E) All the PSR-stained kidneys in the different groups were manually scored in a histological damage scale of 1-10, where 1 represents a normal kidney and 10 a maximally diseased kidney as shown in FIG. 4A-C with special consideration to epithelial integrity. The groups were non-operated or healthy kidneys=Non Op (n=4), UUO diseased kidneys treated with vehicle (n=8), 2 mg/kg 315FST-Fab (n=8), 10 mg/kg 315FST(HBM)-Fab (n=6), 2 mg/kg 315FST(HBM)-Fab (n=8), 0.2 mg/kg 315FST(HBM)-Fab (n=8) and 0.02 mg/kg 315FST(HBM)-Fab (n=8); Statistics were performed using the ordinary one-way ANOVA with uncorrected Fisher's LSD multiple comparison test with NS denoting non-significance, * p<0.05, ** p<0.01, *** p<0.001 and **** p<0.0001. (FIG. 4F) The mice in the different groups described in FIG. 4A-E were monitored throughout the in-life phase for changes in body weight and this was plotted over time for the different groups; non-operated and UUO on treatment with vehicle, 315FST-Fab at 2 mg/kg or FST315(HBM)-Fab at 10, 2, 0.2 or 0.02 mg/kg.

FIG. 5: (FIG. 5A) Principle of the assay described in Example 4. (FIG. 5B) Adriamycin treatment induced a breakdown in barrier integrity as measured by increased permeability to FITC-Dx and protection was afforded in the presence of FST315(HBM)-Fab and not a Fab control.

FIG. 6: (FIG. 6A) Relative expression levels of follistatin fusion proteins were determined by protein-G HPLC analysis of harvested CHO supernatants. Values are normalised relative to the FST-Fc expression level. N-Fab-FST=Fab antibody moiety fused to the N-terminal portion of FST; FST-Fab-C=Fab antibody moiety fused to the C-terminal portion of FST; FST-Fc=Fc antibody moiety fused to the C-terminal portion of FST; FST-ScFv=ScFv antibody moiety fused to the C-terminal portion of FST. (FIG. 6B) Relative expression levels of FST288, FST315, FSTWT and FST-HBM (alternatively named HBSM) with a C-terminal fusion partner (respectively named 288 fusions, 315 fusions, WT fusions and HBSM fusions in the Figure). Values were normalised to the expression of the 288 fusions. (FIG. 6C) Total expression values of monomer, values are normalised relative to the FST-fc expression level, n=25. Same legend as for FIG. 2(A).

FIG. 7: (FIG. 7A) Pharmacokinetic profile of FST315WT, FST315-Fab and FST315HBM-Fab, dosed into mice (n=3 mice per group) at 10 mg/kg via an IV administration route and serum samples were monitored for 7 days; (FIG. 7B) Pharmacokinetic profile of FST288WT, FST288-Fab and FST288HBM-Fab dosed into mice (n=3 mice per group) at 10 mg/kg via an IV administration route and serum samples were monitored for 7 days. The pharmacokinetic parameters derived from all the follistatin moieties of the study are summarized in Table 1.

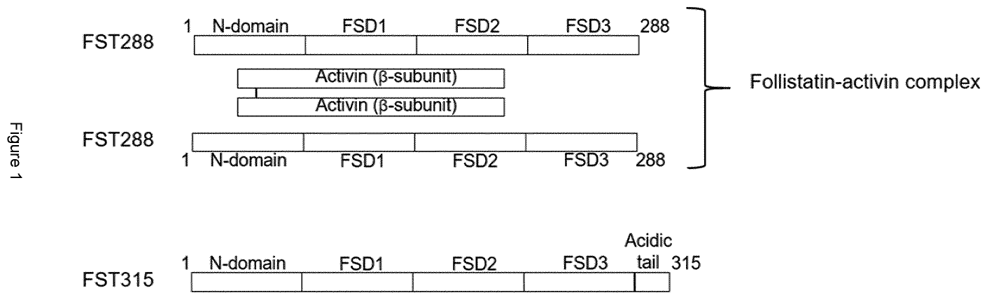

### DETAILED DESCRIPTION OF THE INVENTION

The entire document is intended to be related as a unified disclosure, and it should be understood that all combinations of features described herein are contemplated, even if the combination of features are not found together in the same sentence, or paragraph, or section of this document. With respect to aspects of the invention described or claimed with “a” or “an”, it should be understood that these terms mean “one or more” unless context unambiguously requires a more restricted meaning. The term “or” should be understood to encompass items in the alternative or together unless context unambiguously requires otherwise. If aspects of the invention are described as “comprising” a feature, embodiments also are contemplated “consisting of” or “consisting essentially of” the feature”.

The terms “Follistatin” or “FST”, as used herein, refer to an autocrine glycoprotein (UniProt reference: P19883) which is a known inhibitor of Activins A and B. Follistatin also binds with lower affinity to GDF11, GDF8 (Myostatin), BMPs 2, 4, 6, 7, 11, and 15. There are two main alternatively spliced forms of human Follistatin: a shorter form (FST288, 31.6 kDa) which is cell-bound and a longer circulating form (FST315, 34.8 kDa). The FST315 is defined according to SEQ ID NO: 1 and the FST288 is defined according to SEQ ID NO: 2. The FST315 and 288, have four domains stabilised by a network of disulphide bonds (18 in total), two N-linked glycosylation sites and one heparin binding site. FST315 has an additional 27 amino acids domain (acidic rich) at its C-terminus termed the acidic tail. Also encompassed by the terms are functional fragments and/or functional variants thereof, such as those disclosed in Sidis et al., 2005. If followed by a number, e.g. FST288, this indicates that the protein is the 288 form of follistatin (starting at residue 1 of the mature form). If followed by a number and letters, e.g. FST315HBM, this indicates the heparin-binding mutant (HBM) form as well as the type of variant (here the 315 form of follistatin, starting at residue 1 of the mature form, and including Alanine mutations at residues K76, K81 and K82). Activins are dimeric polypeptide growth factors and belong to the TGF-beta superfamily. Activins can stimulate hormone production in ovarian and placental cells, support neuronal cell survival, and influence cell-cycle progress positively or negatively, depending on cell type. In several tissues, activin signalling is antagonized by its related heterodimer, inhibin. For example, during the release of follicle-stimulating hormone (FSH) from the pituitary, activin promotes FSH secretion and synthesis, while inhibin prevents FSH secretion and synthesis. Activin has also been implicated as a negative regulator of muscle mass and function, and activin antagonists can promote muscle growth or counteract muscle loss in vivo.

The term “antibody” as used herein includes, but is not limited to, monoclonal antibodies, polyclonal antibodies and recombinant antibodies that are generated by recombinant technologies as known in the art. The term “antibody” as used herein includes antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” refers to both glycosylated and aglycosylated antibodies. Furthermore, the term “antibody moiety” as used herein may refer to full-length antibodies, but more generally is intended to reference antibody fragments, and more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). The term “Fab” refers to as used herein refers to an antibody fragment comprising a light chain fragment comprising a VL (variable light) domain and a constant domain of a light chain (CL), and a VH (variable heavy) domain and a first constant domain (CH1) of a heavy chain. The term “Fab′” as employed herein is similar to a Fab, wherein the Fab portion is replaced by a Fab′. The format may be provided as a PEGylated version thereof. Dimers of a Fab′ according to the present disclosure create a F(ab′)2 where, for example, dimerization may be through the hinge. The term “Fv” refers to two variable domains of full-length antibodies, for example co-operative variable domains, such as a cognate pair or affinity matured variable domains, i.e. a VH and VL pair.

The term “single chain variable fragment” or “scFv” as employed herein refers to a single chain variable fragment which is stabilised by a peptide linker between the VH and VL variable domains. The term “single domain antibody” as used herein refers to an antibody fragment consisting of a single monomeric variable domain. Examples of single domain antibodies include VH or VL or VHH or V-NAR.

As used herein, the term “affinity” refers to the strength of all noncovalent interactions between a protein or a fragment thereof and its receptor (if the protein of interest is a ligand) or its ligand (if the protein of interest is a receptor). Unless indicated otherwise, as used herein, the term “binding affinity” refers to intrinsic binding affinity which reflects a 1:1 interaction between members of a binding pair (e.g., a receptor and its ligand). The affinity of a molecule for its binding partner can be generally represented by the dissociation constant (KD). Affinity can be measured by common methods known in the art, including those described herein.

The term “specific” as employed herein in the context of antibodies and antigen-binding fragments is intended to refer to an antibody that only recognizes the antigen to which it is specific or an antibody that has significantly higher binding affinity to the antigen to which it is specific compared to binding to antigens to which it is non-specific, for example at least 5, 6, 7, 8, 9, 10 times higher binding affinity.

The term “chimeric” refers to antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences.

“Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR, i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. Unless indicated otherwise, HVR residues (CDR residues) and other residues in the variable domain (e.g., FR residues) are numbered herein according to Kabat et al.

The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody.

The term “functional variant” as used herein refers to an amino acid sequence which has been modified relative to a reference sequence, but which retains at least one biological function of said reference sequence. For example, a functional variant of FST retains at least one biological activity of the reference FST protein, such as binding and inhibition of Activins A and B.

As used herein, the term “sequence identity” or “identity” refers to the number of matches (identical nucleic acid or amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Alignment for purposes of determining percent nucleic acid or amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

The term “isolated” means, throughout this specification, that the antibody, antigen-binding fragment, polypeptide or polynucleotide, as the case may be, exists in a physical milieu distinct from that in which it may occur in nature. The term “isolated” nucleic acid refers to a nucleic acid molecule that has been isolated from its natural environment or that has been synthetically created. An isolated nucleic acid may comprise synthetic DNA, for instance produced by chemical processing, cDNA, genomic DNA or any combination thereof.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, and/or vehicle with which the therapeutic agent is administered.

As used herein, the terms “treatment”, “treating” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. Treatment thus covers any treatment of a disease in a mammal, particularly in a human, and includes: (a) preventing the disease from occurring in a subject, i.e. a human, which may be predisposed to the disease but has not yet been diagnosed as having it; (b) inhibiting the disease, i.e., arresting its development; and (c) relieving the disease, i.e., causing regression of the disease. The term “therapeutically effective amount” refers to the amount that, when administered to a subject for treating a disease, is sufficient to produce such treatment for the disease.

The term “therapeutically effective amount” refers to the amount that, when administered to a subject for treating a disease, is sufficient to produce such treatment for the disease. The therapeutically effective amount will vary depending on the protein or active fragment thereof, the disease and its severity and the age, weight, etc., of the subject to be treated.

As used herein, “chemotherapy” refers to any agents which, when used alone or in combination, can alleviate, reduce, ameliorate, prevent or otherwise maintain in a state of remission one or more clinical symptoms or diagnostic markers associated with neoplastic disease, tumours and cancer. Exemplary chemotherapy agents include, but are not limited to, alkylating agents, toxins, anticancer antibiotics, antimetabolites, antimitotics, and so on. Particular compounds which are known include, but are not limited to, Ara-C, bleomycin, camptothecin, carboplatin, cisplatin, cyclophosphamide, doxorubicin (Adriamycin), gemcitabine, methotrexate, paclitaxel and vincristine.

The present invention will now be described with respect to particular non-limiting aspects and embodiments thereof and with reference to certain Figures and examples.

The present invention addresses the need to identify effective therapies for use in treatment and prevention of kidney diseases or kidney damage.

The present invention is based on the surprising finding from the inventors that the addition of exogenous follistatin-fusion protein in a number of animal models of kidney disease was associated with a drastic improvement of markers of kidney diseases (such as marker of kidney fibrosis) in said animals. Such markers are for instance hydroxyproline, serum creatinine or collagen. The inventors were then able to surprisingly demonstrate that not only follistatin-fusion proteins can attenuate ECM deposition (as shown by the reduction of collagen) in kidney sections, but also that follistatin-fusion proteins significantly attenuated kidney fibrosis in vivo when administered to UUO mice. It was also surprisingly shown that epithelial cells (such as tubular epithelial cells) in the kidney were protected in the presence of damage stimuli.

Not only is it herein demonstrated that follistatin-fusion proteins can be therapeutic for the treatment and prevention of kidney diseases or kidney damage, but these fusion proteins are more advantageous than previously known follistatin or follistatin-based fusion proteins which comprise an Fc moiety. In particular, the follistatin-fusion proteins that can be used according to the invention are more readily expressed in vitro and exhibit an improved half-life.

The main object of the present invention is a follistatin-fusion protein for use in the treatment, amelioration or prevention of kidney diseases or kidney damage, wherein the follistatin-fusion protein comprises: a. a follistatin moiety, b. an antibody moiety, and optionally c. a linker between the follistatin moiety and the antibody moiety.

The invention also provides a method of treating a patient in need of therapy for treatment, amelioration or prevention of kidney diseases or kidney damage, the method comprising administering a therapeutically effective amount of a follistatin-fusion protein, wherein the follistatin-fusion protein comprises: a. a follistatin moiety, b. an antibody moiety, and optionally c. a linker between the follistatin moiety and the antibody moiety.

Also described is the use of a follistatin-fusion protein in the manufacture of a medicament for the treatment, amelioration or prevention of kidney diseases or kidney damage, wherein the follistatin-fusion protein comprises: a. a follistatin moiety, b. an antibody moiety, and optionally c. a linker between the follistatin moiety and the antibody moiety.

In the context of the present invention as a whole, the kidney disease or the kidney damage is characterised by the variation of certain markers in a subject's sample compared to said markers in a healthy subject's sample, and wherein the sample is a cell or a tissue associated with said disease/damage (e.g. a kidney cell or a kidney tissue) or wherein said sample is for instance (but not limited to) blood, serum or urine. For instance the variation can be an increase of a disease marker, wherein the marker is for instance any one of (but not limited to) serum creatinine, cystatin-C, serum albumin levels, blood urea nitrogen (BUN) or a decrease in estimated glomerular filtration rate (eGFR). Alternatively, the variation can be changes in urinary disease markers such as an increase in albumin or protein, referred to as albuminuria and proteinuria, respectively, and/or an increase in microglobulins, neutrophil gelatinase-associated lipocalin (NGAL) or kidney injury molecule-1 (KIM-1). The variation can also be an increase in one of the follistatin ligands which is linked to disease or progression of disease or damage, which could be (but not limited to) activin A, activin B, GDF8 (myostatin) or GDF11. Said variation of the marker in the sample may be determined by any means. A decrease or an increase of a marker in one subject's sample is typically determined by comparison of the level of said marker in the subject's sample to the level of the same marker in a reference sample (alternatively called here healthy sample or normal sample) of the same sample type (i.e. basal level; e.g. basal disease activity biomarker, basal expression level of follistatin ligands (mRNA level and/or protein level) and/or basal level of fibrotic markers).

The follistatin-fusion protein for use, method, or use of follistatin-fusion protein according to the invention as a whole, e.g. for the treatment, amelioration or prevention of kidney diseases or kidney damage in a subject, may thus comprise the steps of (a) measuring at least one marker of kidney disorders in a sample (e.g. kidney cells, serum or urine) from the subject, wherein the marker is for instance a follistatin ligand activity, follistatin ligand expression or circulating follistatin ligand level, (b) comparing the result of the measure obtained from a) to the corresponding measure in a normal sample, and c) if an increase expression, activity or circulating level of follistatin ligand is observed, administering to the subject said follistatin-fusion protein, thereby treating, ameliorating or preventing the kidney diseases or kidney damage. Follistatin ligand expression that is measured in step a) can be the mRNA or protein amount, and the increase can be any increase of expression as discussed above. The corresponding measure in a normal sample does not need to be obtained each time a comparison is to be made. Said corresponding measure can be obtained any time before the comparison is to be made and can be the average follistatin ligand expression or follistatin ligand activity in a normal cell/tissue.

Alternatively, the follistatin-fusion protein for use, method, or use of follistatin-fusion protein according to the invention as a whole, e.g. for the treatment, amelioration or prevention of kidney diseases or kidney damage in a subject, may thus comprise the steps of (a) measuring at least one marker of kidney disorders in a sample (e.g. kidney cells, serum or urine) from the subject, wherein the marker is for instance proteinuria, serum creatinine or cystatin-C, (b) comparing the result of the measure obtained from a) to the corresponding measure in a normal sample (such as serum or urine), and c) if an increase of expression of said marker is observed, administering to the subject said follistatin-fusion protein, thereby treating, ameliorating or preventing the kidney diseases or kidney damage. Expression of the marker that is measured in step a) can be the mRNA or protein amount, and the increase can be any increase of expression as discussed above. The corresponding measure in a normal sample does not need to be obtained each time a comparison is to be made. Said corresponding measure can be obtained any time before the comparison is to be made and can be the average marker expression in a normal cell/tissue/serum/urine.

In another alternative, the follistatin-fusion protein for use, method, or use of follistatin-fusion protein according to the invention as a whole, e.g. for the treatment, amelioration or prevention of kidney diseases or kidney damage in a subject, may comprise the steps of (a) monitoring the rate of loss of the estimated GFR (eGFR) (and b) if a decrease of eGFR or a measure of the slope of eGFR loss is observed indicative of a fast progressor of kidney function loss (a decrease of >5 ml/min/1.73 m²/year is for instance typically considered accelerated progression), administering to the subject a follistatin-fusion protein, thereby treating, ameliorating or preventing the kidney diseases or kidney damage. Monitoring of eGFR loss can also be considered alongside an increase in proteinuria or albuminuria where values of 30-300 mg/24-hour urine collection are typically considered moderate increases or “microalbuminuria” and increases of >300 mg/24-hour urine collection are typically considered severe or “macroalbuminuria”. The inclusion of albuminuria alongside loss of eGFR is justified as a way of estimating the risk of progression of renal dysfunction and potential risk of ESRF (Ammirati, 2020). Fast progressors of kidney function loss determined by any number of measures could benefit from follistatin-fusion protein treatment as a method of treating, ameliorating or preventing the kidney diseases or kidney damage.

It has to be understood that the markers that can be measured to determine the need of treatment can be combined such as (in a none limiting example) measuring the increase of follistatin ligand expression combined with the increase of serum creatinine or proteinuria or albuminuria.

In the context of the present invention as a whole, the kidney disease is preferably a chronic kidney disease such as (but not limited to) primary glomerulonephritis, secondary glomerulonephritis, diabetic nephropathy, hypertensive nephrosclerosis, focal segmental glomerulosclerosis (FSGS), IgA nephropathy (IgAN), Mesangial proliferative glomerulonephritis, Membranous Nephropathy (MN), Minimal Change Disease (MCD), polycystic kidney disease, chronic allograft nephropathy, CKD-associated mineral and bone disorder (CKD-MBD) and Goodpastures disease. Alternatively, the kidney disease is a chronic kidney disease selected from primary glomerulonephritis, secondary glomerulonephritis, diabetic nephropathy, hypertensive nephrosclerosis, focal segmental glomerulosclerosis (FSGS), IgA nephropathy (IgAN), Mesangial proliferative glomerulonephritis, Membranous Nephropathy (MN), Minimal Change Disease (MCD), polycystic kidney disease, chronic allograft nephropathy, CKD-associated mineral and bone disorder (CKD-MBD) and Goodpastures disease. Also in the context of the present invention as a whole, the kidney damage is a) kidney fibrosis, or b) is associated with other diseases such as diabetes mellitus, hypertension, cardiovascular disease or bone disorder. Both primary and secondary forms of glomerulonephritis can progress to end stage renal failure (ESRF), as can most forms of renal disease. Conventional treatment calls for dialysis, but outcomes are generally poor, with a survival rate of less than 50% after five or more years. Renal transplant is currently considered to be the gold standard treatment, but organ recipient rates are low. The kidney disease or kidney damage can be a disease or a damage on its own or can be the result of a therapeutic treatment, such as a treatment with chemotherapy. In this context, the chemotherapy agent can be selected from, but not limited to, alkylating agents, toxins, anticancer antibiotics, antimetabolites, antimitotics, and so on. Particular compounds which are known to cause kidney damage include, but are not limited to, Ara-C, bleomycin, camptothecin, carboplatin, cisplatin, cyclophosphamide, doxorubicin (Adriamycin), gemcitabine, methotrexate, paclitaxel and vincristine. When the follistatin-fusion protein is administered in the context of a kidney disease or kidney damage that is/could be the result of a therapeutic treatment said fusion protein can be administered simultaneously, separately or sequentially with the other treatment, such as a chemotherapy. In a particular example, the follistatin-fusion protein according to the invention is used to treat, prevent or ameliorate a kidney disease or kidney damage resulting from treatment with doxorubicin (Adriamycin).

Without wishing to be bound by any theory, the present inventors found that a follistatin-based therapy using the fusion protein of the invention can modulate ligand signalling, leading to increased epithelial cell (such as tubular epithelial cell) protection and reduced pro-fibrotic signalling which will reduce scar tissue in the kidney. Accordingly, this treatment could slow or prevent tissue damage and fibrosis associated with progressive renal disease, leading to a reduction in eGFR loss and a reduction in proteinuria. Prevention of end-stage kidney failure and/or extension of time to renal replacement therapy/transplantation can thereby be achieved. In other words, the treatment, amelioration or prevention of kidney diseases or kidney damage in a subject in the context of the invention as a whole is obtained via protection of the epithelial cells (such as tubular epithelial cells).

The follistatin moiety of the follistatin-fusion protein to be used in the context of the present invention as a whole comprises or is a naturally occurring follistatin protein. It is preferably a mature form thereof, i.e. lacking the N-secretion signal sequence as this sequence is needed only for the production/secretion from a cell. Alternatively, it is a functional fragment thereof. Said follistatin moiety is for instance the FST288 protein (SEQ ID NO.2) or the FST315 protein (SEQ ID NO.1). Any intermediate forms thereof, e.g. any follistatin moieties comprising between 289 and 314 residues of any one of SEQ ID NOs. 1 to 4, can also be used, as long as they are functional, i.e. that they retain at least one biological activity of FST. Although none limiting, preferably any intermediate forms of the follistatin moieties start at residue 1 of SEQ ID NO:1. As none limiting examples, the functional FST fragment can be FST291 (i.e. comprising residues 1 to 291 of SEQ ID No.1) or FST303 (i.e. comprising residues 1 to 303 of SEQ ID No.1). In another alternative, the follistatin moiety according to the invention (i.e. naturally occurring or functional fragment thereof) is a functional variant, e.g. it can have one or more mutations, such as mutations in the heparin binding site (HBS). As none limiting example, the one or more mutation sites can be selected from K76, K81 and/or K82 numbered relative to SEQ ID NO: 1 (see sequences 22 and 25 as examples). The one or more mutations may comprise Alanine (A) in place of Lysine (K) (resulting in mutations selected from K76A, K81A and/or K82A). As a further none limiting example, a heparin binding mutant (“HBM”)(alternatively herein named “(HBM)” or “HBSM”) can be used, e.g. FST288HBM (SEQ ID NO: 4), FST291 HBM, FST303HBM or FST315HBM (SEQ ID NO: 3), wherein said mutant comprises the triple mutations K76A, K81A and K82A. Preferably, the follistatin moiety is selected from: a. SEQ ID NO: 1; b. SEQ ID NO: 2; c. SEQ ID NO: 3; d. SEQ ID NO: 4; e. any protein comprising amino acid residues comprising between 289 and 314 residues of any one of SEQ ID Nos.1 to 4; or f. a sequence having at least 95%, at least 96%, at least 97%, at least 98% or at least 99% sequence identity to any one of SEQ ID NOs: 1 to 4.

The antibody moiety of the follistatin-fusion protein to be used in the context of the present invention as a whole binds albumin, and preferably binds serum albumin, such as human serum albumin (HSA). Said antibody moiety can be a chimeric, humanized or human antibody moiety. Preferably, the antibody moiety is an antigen-binding fragment of an antibody (alternatively herein called antigen-binding moiety). Preferably it is selected from a Fab, a Fab′ or a F(ab′)2. In a none limiting example, the antibody moiety comprises a light chain variable region comprising a CDR-L1 comprising SEQ ID NO: 13; a CDR-L2 comprising SEQ ID NO: 14 and a CDR-L3 comprising SEQ ID NO: 15; and a heavy chain variable region comprising a CDR-H1 comprising SEQ ID NO: 16; a CDR-H2 comprising SEQ ID NO: 17 and/or a CDR-H3 comprising SEQ ID NO: 18. In an alternative none limiting example, the antibody moiety comprises a heavy chain variable region comprising or consisting of SEQ ID NO: 5, or a sequence having at least 95%, at least 96%, at least 97%, at least 98% or at least 99% sequence identity thereto; and a light chain variable region comprising or consisting of SEQ ID NO: 6, or a sequence having at least 95%, at least 96%, at least 97%, at least 98% or at least 99% sequence identity thereto. SEQ ID NO: 5 and SEQ ID NO: 6 represent the heavy and light variable chains of an anti-albumin antibody designated “CA645” (as disclosed in WO2013/068571).

In the context of the invention as a whole, the follistatin-fusion proteins optionally comprise a linker between the follistatin moiety and the antibody moiety. When the linker is present, as none limiting examples it can be selected from the group consisting of: SGGGGS (SEQ ID NO: 7), SGGGGSSGGGGS (SEQ ID NO: 19), GGGGS (SEQ ID NO: 20) and GGGGSGGGGS (SEQ ID NO: 21).

It will be understood that when creating a fusion protein, there are two options for fusing/combining any moieties to each other: C-terminal fusion or N-terminal fusion. It was found by the inventors that fusing the N-terminal moiety of the antibody moiety to the C-terminal portion of the follistatin moiety resulted in yet further improved expression of the resulting fusion protein, by comparison with any other type of fusions, In particular, the C-terminal fusion proteins of the invention exhibit superior expression and higher yield of monomeric protein by comparison with the known Fc-based follistatin fusion proteins. However, although the C-terminal fusion proteins resulted in the highest expression level, antibody moiety fused to the N-terminal of follistatin could be considered by the skilled persons as they result for instance in about 1.5 higher expression level compared to Fc-based follistatin fusion proteins. Accordingly, in a preferred embodiment, the antibody moiety is connected to the C-terminal portion of the follistatin moiety. If a linker is present, the antibody moiety is preferably connected (or conjugated), via the linker, to the C-terminal portion of the follistatin moiety (in other words, the antibody moiety is connected (or conjugated) to the C-terminal portion of the follistatin moiety, and there is a linker between the two moieties).

Similarly, it will be understood that when creating a fusion protein between one polypeptide (here an FST moiety) and an antibody moiety (here preferably a Fab, Fab′ or F(ab′)2), there are two options for fusing any moieties to each other: either the polypeptide is fused to the heavy chain of the antibody moiety or to the light chain of the antibody moiety.

None limiting examples of follistatin-fusion proteins that can be used according to the invention are:
(a) i. an FST315 polypeptide defined by SEQ ID NO: 1, or a sequence having at least 95%, at least 96%, at least 97%, at least 98% or at least 99% sequence identity thereto;
   ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide; and
   iii. a Fab light chain defined by SEQ ID NO: 6, or a sequence having at least 95%, at least 96%, at least 97%, at least 98% or at least 99% sequence identity thereto;
(b) i. an FST288 polypeptide defined by SEQ ID NO: 2, or a sequence having at least 95%, at least 96%, at least 97%, at least 98% or at least 99% sequence identity thereto;
   ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide; and
   iii. a Fab light chain defined by SEQ ID NO: 6, or a sequence having at least 95%, at least 96%, at least 97%, at least 98% or at least 99% sequence identity thereto;
(c) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
   ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide variant; and
   iii. a Fab light chain defined by SEQ ID NO: 6, or a sequence having at least 95%, at least 96%, at least 97%, at least 98% or at least 99% sequence identity thereto;
(d) i. an FST288 polypeptide variant defined by SEQ ID NO: 4 (FST288HBM) or SEQ ID NO: 25, or a sequence having at least 95%, at least 96%, at least 97%, at least 98% or at least 99% sequence identity thereto;
   ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide variant; and
   iii. a Fab light chain defined by SEQ ID NO: 6, or a sequence having at least 95%, at least 96%, at least 97%, at least 98% or at least 99% sequence identity thereto;
(e) i. an FST315 polypeptide defined by SEQ ID NO: 1, or a sequence having at least 95%, at least 96%, at least 97%, at least 98% or at least 99% sequence identity thereto;
   ii. a linker defined by SEQ ID NO: 7, SEQ ID NO: 19, SEQ ID NO: 20 or SEQ ID NO: 21 conjugated to the C-terminus of the FST315 polypeptide;
   iii. a Fab heavy chain defined by SEQ ID NO: 5, or a sequence having at least 95%, at least 96%, at least 97%, at least 98% or at least 99% sequence identity thereto conjugated
   to the free-terminus of the linker; and iv. a Fab light chain defined by SEQ ID NO: 6, or a sequence having at least 95%, at least 96%, at least 97%, at least 98% or at least 99% sequence identity thereto;
(f) i. an FST288 polypeptide defined by SEQ ID NO: 2, or a sequence having at least 95%, at least 96%, at least 97%, at least 98% or at least 99% sequence identity thereto;
   ii. a linker defined by SEQ ID NO: 7, SEQ ID NO: 19, SEQ ID NO: 20 or SEQ ID NO: 21 conjugated to the C-terminus of the FST288 polypeptide;
   iii. a Fab heavy chain defined by SEQ ID NO: 5, or a sequence having at least 95%, at least 96%, at least 97%, at least 98% or at least 99% sequence identity thereto conjugated to the free-terminus of the linker; and
   iv. a Fab light chain defined by SEQ ID NO: 6, or a sequence having at least 95%, at least 96%, at least 97%, at least 98% or at least 99% sequence identity thereto;
(g) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
   ii. a linker defined by SEQ ID NO: 7, SEQ ID NO: 19, SEQ ID NO: 20 or SEQ ID NO: 21 conjugated to the C-terminus of the FST315 polypeptide variant;
   iii. a Fab heavy chain defined by SEQ ID NO: 5, or a sequence having at least 95%, at least 96%, at least 97%, at least 98% or at least 99% sequence identity thereto conjugated to the free-terminus of the linker; and
   iv. a Fab light chain defined by SEQ ID NO: 6, or a sequence having at least 95%, at least 96%, at least 97%, at least 98% or at least 99% sequence identity thereto;
(h) i. an FST288 polypeptide variant defined by SEQ ID NO: 4 (FST288HBM) or SEQ ID NO:25, or a sequence having at least 95%, at least 96%, at least 97%, at least 98% or at least 99% sequence identity thereto;
   ii. a linker defined by SEQ ID NO: 7, SEQ ID NO: 19, SEQ ID NO: 20 or SEQ ID NO: 21 conjugated to the C-terminus of the FST288 polypeptide variant;
   iii. a Fab heavy chain defined by SEQ ID NO: 5, or a sequence having at least 95%, at least 96%, at least 97%, at least 98% or at least 99% sequence identity thereto conjugated to the free-terminus of the linker; and
   iv. a Fab light chain defined by SEQ ID NO: 6, or a sequence having at least 95%, at least 96%, at least 97%, at least 98% or at least 99% sequence identity thereto;
(i) SEQ ID NO: 8, 9, 10, 11, 24, 25, 26, 27, 32, 33, 34 or 35, or a sequence having at least 95%, at least 96%, at least 97%, at least 98% or at least 99% sequence identity thereto, together with a Fab light chain defined by SEQ ID NO: 6 or a sequence having at least 95%, at least 96%, at least 97%, at least 98% or at least 99% sequence identity thereto;
(j) SEQ ID NO: 8, 9, 10, 11, 24, 25, 26, 27, 32, 33, 34 or 35, together with a Fab light chain defined by SEQ ID NO: 6; or
(k) a functional variant or fragment of any one of (a) to (i).

Any subject may be treated in accordance with the invention. The subject is preferably human. However, the subject may be another mammalian animal, such as a non-human primate, a horse, a cow, a sheep, a pig, a dog, a cat, a rabbit, a rat, a mouse, a guinea pig or a hamster. Alternatively, the term patient can be used indifferently instead of subject.

Any follistatin-fusion protein to be used according to the invention may be incorporated into pharmaceutical compositions suitable for administration to a subject in any way, such as (but not limited to) topically, intranasally, intradermally, intravenously, subcutaneously or intramuscularly. Typically, the pharmaceutical composition to be used according to the invention comprises a follistatin-fusion protein and one or more pharmaceutically acceptable adjuvant(s) and/or carrier(s). Therefore, herein described is also a pharmaceutical composition for use in the treatment, amelioration or prevention of kidney diseases or kidney damage in a subject, wherein said pharmaceutical composition comprises follistatin-fusion protein as herein described and one or more pharmaceutically acceptable adjuvant(s) and/or carrier(s). The pharmaceutical composition according to the invention can be part of a kit with instructions for use, including instructions and optionally a device for intravenous, subcutaneous or intramuscular administration to the individual in need thereof.

As used herein, “pharmaceutically acceptable carrier” includes any and all solvents, dispersion media, coatings, antibacterial and antifungal agents, isotonic and absorption delaying agents, and the like that are physiologically compatible and are suitable for administration to a subject for the methods and uses described herein. Examples of pharmaceutically acceptable carriers include one or more of water, saline, buffered saline, dextrose, glycerol, ethanol and the like, as well as combinations thereof. Depending on the route of administration or the type of formulation (such as liquid, freeze-dried or spray-dried formulation), isotonic agents can be incorporated, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The pharmaceutical compositions according to the present invention may be in a variety of forms. These include, for example, liquid solutions (e.g., injectable and infusible solutions), dispersions or suspensions, powders and liposomes. The preferred form depends on the intended mode of administration and therapeutic application. Typical preferred compositions are in the form of injectable or infusible solutions, such as compositions similar to those used for passive immunization of humans with other antibodies.

A suitable dosage of a follistatin-fusion protein according to the present invention may be determined by a skilled medical practitioner. Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to said patient. The selected dosage level will depend upon a variety of pharmacokinetic factors including the route of administration, the time of administration, the rate of excretion of the follistatin-fusion protein, the duration of the treatment, other drugs, compounds and/or materials used in combination with the particular follistatin-fusion protein, the age, sex, weight, condition, general health and prior medical history of the patient being treated.

A suitable dose may be, for example, in the range of from about 0.01 μg/kg to about 1000 mg/kg body weight, typically from about 0.1 pg/kg to about 100 mg/kg body weight, of the patient to be treated. Dosage regimens may be adjusted to provide the optimum desired response (e.g. a therapeutic response). For example, a single dose may be administered, or several divided doses may be administered over time. Dosage unit form as used herein refers to physically discrete units suited as unitary dosages for the subjects to be treated; each unit contains a predetermined quantity of active compound calculated to produce the desired therapeutic effect in association with the required pharmaceutical earner. Administration may be in single or multiple doses. Multiple doses may be administered via the same or different routes and to the same or different locations. In the context of the invention as a whole the follistatin-fusion protein may be co-administered with one or other more other therapeutic agents. Combined administration of two or more agents may be achieved in a number of different ways. Both may be administered together in a single composition, or they may be administered in separate compositions as part of a combined therapy. For example, the one may be administered before or separately, after or sequential, or concurrently or simultaneously with the other. In particular if the kidney disease or kidney damage is/can be the result of a chemotherapy, the chemotherapy agent can be selected from, but not limited to, alkylating agents, toxins, anticancer antibiotics, antimetabolites, antimitotics, and so on. Particular compounds which are known include, but are not limited to, Ara-C, bleomycin, camptothecin, carboplatin, cisplatin, cyclophosphamide, doxorubicin (Adriamycin), gemcitabine, methotrexate, paclitaxel and vincristine. In a particular example, the follistatin-fusion protein or pharmaceutical composition according to the invention is used to treat, prevent or ameliorate kidney diseases or kidney damage resulting from treatment with doxorubicin (Adriamycin).

It should be noted that the above-mentioned embodiments illustrate rather than limit the invention, and that those skilled in the art will be able to design many alternative embodiments without departing from the scope of the invention and in particular of the claims.

### DESCRIPTION OF THE SEQUENCES

| | | SEQ |
|---|---|---|
| Name | Sequence | ID NO |
| FST315 - mature | GNCWLRQAKNGRCQVLYKTELSKEECCSTGRLSTSWTEEDVN | 1 |
| form | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEW | |
| FST288 - mature | GNCWLRQAKNGRCQVLYKTELSKEECCSTGRLSTSWTEEDVN | 2 |
| form (i.e. lacking N- | DNTLFKWMIFNGGAPNCIPCKETCENVDCGPGKKCRMNKKNKP | |
| terminal secretion | RCVCAPDCSNITWKGPVCGLDGKTYRNECALLKARCKEQPELE | |
| signal peptide) | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCN | |
| FST315HBM - | GNCWLRQAKNGRCQVLYKTELSKEECCSTGRLSTSWTEEDVN | 3 |
| mature form | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEW | |
| FST288HBM - | GNCWLRQAKNGRCQVLYKTELSKEECCSTGRLSTSWTEEDVN | 4 |
| mature form | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCN | |
| Fab heavy chain | EVQLLESGGGLVQPGGSLRLSCAVSGIDLSNYAINWVRQAPGK | 5 |
| (FabHC) | GLEWIGIIWASGTTFYATWAKGRFTISRDNSKNTVYLQMNSLRA | |
| | EDTAVYYCARTVPGYSTAPYFDLWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSC | |
| Fab light chain | DIQMTQSPSSVSASVGDRVTITCQSSPSVWSNFLSWYQQK | 6 |
| (FabLC) | PGKAPKLLIYEASKLTSGVPSRFSGSGSGTDFTLTISSLOPEDFA | |
| | TYYCGGGYSSISDTTFGGGTKVEIKRTVAAPSVFIFPPSDEQLKS | |
| | GTASVVCLLNNFYPREAKVQWKVDNALQSGNSQESVTEQDSK | |
| | DSTYSLSSTLTLSKADYEKHKVYACEVTHQGLSSPVTKSFNRGE | |
| | C | |
| Exemplary Linker | SGGGGS | 7 |
| FST315HBM-FabHC | GNCWLRQAKNGRCQVLYKTELSKEECCSTGRLSTSWTEEDVN | 8 |
| (with linker | DNTLFKWMIFNGGAPNCIPCKETCENVDCGPGKACRMNAANKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWSGGGGSEVQLLESGGGLVQPGGSLRLSC | |
| | AVSGIDLSNYAINWVRQAPGKGLEWIGIIWASGTTFYATWAKGR | |
| | FTISRDNSKNTVYLQMNSLRAEDTAVYYCARTVPGYSTAPYFDL | |
| | WGQGTLVTVSSASTKGPSVFPLAPSSKSTSGGTAALGCLVKDY | |
| | FPEPVTVSWNSGALTSGVHTFPAVLQSSGLYSLSSVVTVPSSSL | |
| | GTQTYICNVNHKPSNTKVDKKVEPKSC | |
| FST315HBM-FabHC | GNCWLRQAKNGRCQVLYKTELSKEECCSTGRLSTSWTEEDVN | 9 |
| (without linker) | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWEVQLLESGGGLVQPGGSLRLSCAVSGIDL | |
| | SNYAINWVRQAPGKGLEWIGIIWASGTTFYATWAKGRFTISRDN | |
| | SKNTVYLQMNSLRAEDTAVYYCARTVPGYSTAPYFDLWGQGTL | |
| | VTVSSASTKGPSVFPLAPSSKSTSGGTAALGCLVKDYFPEPVTV | |
| | SWNSGALTSGVHTFPAVLQSSGLYSLSSVVTVPSSSLGTQTYIC | |
| | NVNHKPSNTKVDKKVEPKSC | |
| FST288HBM-FabHC | GNCWLRQAKNGRCQVLYKTELSKEECCSTGRLSTSWTEEDVN | 10 |
| (with linker | DNTLFKWMIFNGGAPNCIPCKETCENVDCGPGKACRMNAANKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSGGGGSEVQLL | |
| | ESGGGLVQPGGSLRLSCAVSGIDLSNYAINWVRQAPGKGLEWI | |
| | GIIWASGTTFYATWAKGRFTISRDNSKNTVYLQMNSLRAEDTAV | |
| | YYCARTVPGYSTAPYFDLWGQGTLVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKV | |
| | EPKSC | |
| FST288HBM-FabHC | GNCWLRQAKNGRCQVLYKTELSKEECCSTGRLSTSWTEEDVN | 11 |
| (without linker ) | DNTLFKWMIFNGGAPNCIPCKETCENVDCGPGKACRMNAANKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNEVQLLESGGGLV | |
| | QPGGSLRLSCAVSGIDLSNYAINWVRQAPGKGLEWIGIIWASGT | |
| | TFYATWAKGRFTISRDNSKNTVYLQMNSLRAEDTAVYYCARTV | |
| | PGYSTAPYFDLWGQGTLVTVSSASTKGPSVFPLAPSSKSTSGG | |
| | TAALGCLVKDYFPEPVTVSWNSGALTSGVHTFPAVLQ | |
| | SSGLYSLSSVVTVPSSSLGTQTYICNVNHKPSNTKVDKKV | |
| | EPKSC | |
| N-terminal secretion | MVRARHQPGGLCLLLLLLCQFMEDRSAQA | 12 |
| signal sequence/ | | |
| peptide | | |
| Light Chain CDR1 | QSSPSVWSNFLS | 13 |
| Light Chain CDR2 | EASKLTS | 14 |
| Light Chain CDR3 | GGGYSSISDTT | 15 |
| Heavy Chain CDR1 | GIDLSNYAIN | 16 |
| Heavy Chain CDR2 | IIWASGTTFYATWAKG | 17 |
| Heavy Chain CDR3 | TVPGYSTAPYFDL | 18 |
| Alternative | SGGGGSSGGGGS | 19 |
| Exemplary Linker | | |
| Alternative | GGGGS | 20 |
| Exemplary Linker | | |
| Alternative | GGGGSGGGGS | 21 |
| Exemplary Linker | | |
| FST315(variant) | GNCWLRQAKNGRCQVLYKTELSKEECCSTGRLSTSWTEEDVN | 22 |
| | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEW | |
| FST288(variant) | GNCWLRQAKNGRCQVLYKTELSKEECCSTGRLSTSWTEEDVN | 23 |
| | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCN | |
| FST315(variant)- | GNCWLRQAKNGRCQVLYKTELSKEECCSTGRLSTSWTEEDVN | 24 |
| FabHC | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWEVOLLESGGGLVQPGGSLRLSCAVSGIDL | |
| | SNYAINWVRQAPGKGLEWIGIIWASGTTFYATWAKGRFTISRDN | |
| | SKNTVYLQMNSLRAEDTAVYYCARTVPGYSTAPYFDLWGQGTL | |
| | VTVSSASTKGPSVFPLAPSSKSTSGGTAALGCLVKDYFPEPVTV | |
| | SWNSGALTSGVHTFPAVLQSSGLYSLSSVVTVPSSSLGTQTYIC | |
| | NVNHKPSNTKVDKKVEPKSC | |
| FST315(variant)- | GNCWLRQAKNGRCQVLYKTELSKEECCSTGRLSTSWTEEDVN | 25 |
| FabHC (with linker | DNTLFKWMIFNGGAPNCIPCKETCENVDCGPGKXCRMNXXNKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWSGGGGSEVQLLESGGGLVQPGGSLRLSC | |
| | AVSGIDLSNYAINWVRQAPGKGLEWIGIIWASGTTFYATWAKGR | |
| | FTISRDNSKNTVYLQMNSLRAEDTAVYYCARTVPGYSTAPYFDL | |
| | WGQGTLVTVSSASTKGPSVFPLAPSSKSTSGGTAALGCLVKDY | |
| | FPEPVTVSWNSGALTSGVHTFPAVLQSSGLYSLSSVVTVPSSSL | |
| | GTQTYICNVNHKPSNTKVDKKVEPKSC | |
| FST288(variant)- | GNCWLRQAKNGRCQVLYKTELSKEECCSTGRLSTSWTEEDVN | 26 |
| FabHC | DNTLFKWMIFNGGAPNCIPCKETCENVDCGPGKXCRMNXXNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNEVQLLESGGGLV | |
| | QPGGSLRLSCAVSGIDLSNYAINWVRQAPGKGLEWIGIIWASGT | |
| | TFYATWAKGRFTISRDNSKNTVYLQMNSLRAEDTAVYYCARTV | |
| | PGYSTAPYFDLWGQGTLVTVSSASTKGPSVFPLAPSSKSTSGG | |
| | TAALGCLVKDYFPEPVTVSWNSGALTSGVHTFPAVLQSSGLYSL | |
| | SSVVTVPSSSLGTQTYICNVNHKPSNTKVDKKVEPKSC | |
| FST288(variant)- | GNCWLRQAKNGRCQVLYKTELSKEECCSTGRLSTSWTEEDVN | 27 |
| FabHC (with linker | DNTLFKWMIFNGGAPNCIPCKETCENVDCGPGKXCRMNXXNKP | |
| underlined) | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSGGGGSEVQLL | |
| | ESGGGLVQPGGSLRLSCAVSGIDLSNYAINWVRQAPGKGLEWI | |
| | GIIWASGTTFYATWAKGRFTISRDNSKNTVYLQMNSLRAEDTAV | |
| | YYCARTVPGYSTAPYFDLWGQGTLVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKVEPK | |
| | SC | |
| FabHC- | EVQLLESGGGLVQPGGSLRLSCAVSGIDLSNYAINWVRQAPGK | 28 |
| FST315(variant) | GLEWIGIIWASGTTFYATWAKGRFTISRDNSKNTVYLQMNSLRA | |
| | EDTAVYYCARTVPGYSTAPYFDLWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCGNCWLRQAKNGRCQVLYKTELSKEECCSTGRLSTS | |
| | WTEEDVNDNTLFKWMIFNGGAPNCIPCKETCENVDCGPGKXC | |
| | RMNXXNKPRCVCAPDCSNITWKGPVCGLDGKTYRNECALLKA | |
| | RCKEQPELEVQYQGRCKKTCRDVFCPGSSTCVVDQTNNAYCV | |
| | TCNRICPEPASSEQYLCGNDGVTYSSACHLRKATCLLGRSIGLA | |
| | YEGKCIKAKSCEDIQCTGGKKCLWDFKVGRGRCSLCDELCPDS | |
| | KSDEPVCASDNATYASECAMKEAACSSGVLLEVKHSGSCNSIS | |
| | EDTEEEEEDEDQDYSFPISSILEW | |
| FabHC- | EVQLLESGGGLVQPGGSLRLSCAVSGIDLSNYAINWVRQAPGK | 29 |
| FST315(variant)- | GLEWIGIIWASGTTFYATWAKGRFTISRDNSKNTVYLQMNSLRA | |
| (with linker | EDTAVYYCARTVPGYSTAPYFDLWGQGTLVTVSSASTKGPSVF | |
| underlined) | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCSGGGGSGNCWLRQAKNGRCQVLYKTELSKEECCST | |
| | GRLSTSWTEEDVNDNTLFKWMIFNGGAPNCIPCKETCENVDCG | |
| | PGKXCRMNXXNKPRCVCAPDCSNITWKGPVCGLDGKTYRNEC | |
| | ALLKARCKEQPELEVQYQGRCKKTCRDVFCPGSSTCVVDQTN | |
| | NAYCVTCNRICPEPASSEQYLCGNDGVTYSSACHLRKATCLLG | |
| | RSIGLAYEGKCIKAKSCEDIQCTGGKKCLWDFKVGRGRCSLCD | |
| | ELCPDSKSDEPVCASDNATYASECAMKEAACSSGVLLEVKHSG | |
| | SCNSISEDTEEEEEDEDQDYSFPISSILEW | |
| FabHC- | EVQLLESGGGLVQPGGSLRLSCAVSGIDLSNYAINWVRQAPGK | 30 |
| FST288(variant) | GLEWIGIIWASGTTFYATWAKGRFTISRDNSKNTVYLQMNSLRA | |
| | EDTAVYYCARTVPGYSTAPYFDLWGQGTLVTVSSASTKGPSVF | |
| | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCGNCWLRQAKNGRCQVLYKTELSKEECCSTGRLSTS | |
| | WTEEDVNDNTLFKWMIFNGGAPNCIPCKETCENVDCGPGKXC | |
| | RMNXXNKPRCVCAPDCSNITWKGPVCGLDGKTYRNECALLKA | |
| | RCKEQPELEVQYQGRCKKTCRDVFCPGSSTCVVDQTNNAYCV | |
| | TCNRICPEPASSEQYLCGNDGVTYSSACHLRKATCLLGRSIGLA | |
| | YEGKCIKAKSCEDIQCTGGKKCLWDFKVGRGRCSLCDELCPDS | |
| | KSDEPVCASDNATYASECAMKEAACSSGVLLEVKHSGSCN | |
| FabHC- | EVQLLESGGGLVQPGGSLRLSCAVSGIDLSNYAINWVRQAPGK | 31 |
| FST288(variant)- | GLEWIGIIWASGTTFYATWAKGRFTISRDNSKNTVYLQMNSLRA | |
| (with linker | EDTAVYYCARTVPGYSTAPYFDLWGQGTLVTVSSASTKGPSVF | |
| underlined) | PLAPSSKSTSGGTAALGCLVKDYFPEPVTVSWNSGALTSGVHT | |
| | FPAVLQSSGLYSLSSVVTVPSSSLGTQTYICNVNHKPSNTKVDK | |
| | KVEPKSCSGGGGSGNCWLRQAKNGRCQVLYKTELSKEECCST | |
| | GRLSTSWTEEDVNDNTLFKWMIFNGGAPNCIPCKETCENVDCG | |
| | PGKXCRMNXXNKPRCVCAPDCSNITWKGPVCGLDGKTYRNEC | |
| | ALLKARCKEQPELEVQYQGRCKKTCRDVFCPGSSTCVVDQTN | |
| | NAYCVTCNRICPEPASSEQYLCGNDGVTYSSACHLRKATCLLG | |
| | RSIGLAYEGKCIKAKSCEDIQCTGGKKCLWDFKVGRGRCSLCD | |
| | ELCPDSKSDEPVCASDNATYASECAMKEAACSSGVLLEVKHSG | |
| | SCN | |
| FST315-FabHC (with | GNCWLRQAKNGRCQVLYKTELSKEECCSTGRLSTSWTEEDVN | 32 |
| linker underlined) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWSGGGGSEVQLLESGGGLVQPGGSLRLSC | |
| | AVSGIDLSNYAINWVRQAPGKGLEWIGIIWASGTTFYATWAKGR | |
| | FTISRDNSKNTVYLQMNSLRAEDTAVYYCARTVPGYSTAPYFDL | |
| | WGQGTLVTVSSASTKGPSVFPLAPSSKSTSGGTAALGCLVKDY | |
| | FPEPVTVSWNSGALTSGVHTFPAVLQSSGLYSLSSVVTVPSSSL | |
| | GTQTYICNVNHKPSNTKVDKKVEPKSC | |
| FST315-FabHC | GNCWLRQAKNGRCQVLYKTELSKEECCSTGRLSTSWTEEDVN | 33 |
| (without linker) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSISEDTEEEEED | |
| | EDQDYSFPISSILEWEVQLLESGGGLVQPGGSLRLSCAVSGIDL | |
| | SNYAINWVRQAPGKGLEWIGIIWASGTTFYATWAKGRFTISRDN | |
| | SKNTVYLQMNSLRAEDTAVYYCARTVPGYSTAPYFDLWGQGTL | |
| | VTVSSASTKGPSVFPLAPSSKSTSGGTAALGCLVKDYFPEPVTV | |
| | SWNSGALTSGVHTFPAVLQSSGLYSLSSVVTVPSSSLGTQTYIC | |
| | NVNHKPSNTKVDKKVEPKSC | |
| FST288-FabHC (with | GNCWLRQAKNGRCQVLYKTELSKEECCSTGRLSTSWTEEDVN | 34 |
| linker underlined) | DNTLFKWMIFNGGAPNCIPCKETCENVDCGPGKKCRMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNSGGGGSEVQLL | |
| | ESGGGLVQPGGSLRLSCAVSGIDLSNYAINWVRQAPGKGLEWI | |
| | GIIWASGTTFYATWAKGRFTISRDNSKNTVYLQMNSLRAEDTAV | |
| | YYCARTVPGYSTAPYFDLWGQGTLVTVSSASTKGPSVFPLAPS | |
| | SKSTSGGTAALGCLVKDYFPEPVTVSWNSGALTSGVHTFPAVL | |
| | QSSGLYSLSSVVTVPSSSLGTQTYICNVNHKPSNTKVDKKVEPK | |
| | SC | |
| FST288-FabHC | GNCWLRQAKNGRCQVLYKTELSKEECCSTGRLSTSWTEEDVN | 35 |
| (without linker) | DNTLFKWMIFNGGAPNCIPCKETCENVDCGPGKKORMNKKNKP | |
| | RCVCAPDCSNITWKGPVCGLDGKTYRNECALLKARCKEQPELE | |
| | VQYQGRCKKTCRDVFCPGSSTCVVDQTNNAYCVTCNRICPEPA | |
| | SSEQYLCGNDGVTYSSACHLRKATCLLGRSIGLAYEGKCIKAKS | |
| | CEDIQCTGGKKCLWDFKVGRGRCSLCDELCPDSKSDEPVCAS | |
| | DNATYASECAMKEAACSSGVLLEVKHSGSCNEVQLLESGGGLV | |
| | QPGGSLRLSCAVSGIDLSNYAINWVRQAPGKGLEWIGIIWASGT | |
| | TFYATWAKGRFTISRDNSKNTVYLQMNSLRAEDTAVYYCARTV | |
| | PGYSTAPYFDLWGQGTLVTVSSASTKGPSVFPLAPSSKSTSGG | |
| | TAALGCLVKDYFPEPVTVSWNSGALTSGVHTFPAVLQSSGLYSL | |
| | SSVVTVPSSSLGTQTYICNVNHKPSNTKVDKKVEPKSC | |

* The Xs in SEQ ID Nos 22-31 stand “for any amino acid”

### EXAMPLES

The following examples are merely illustrative of the invention and are not intended to limit the scope or content of the invention.

### Materials & Methods

Cloning strategy: DNA segments corresponding to fusions between follistatin and anti-albumin antibody (designated 645 Fab) heavy or light chain sequences (with or without linker sequences in between) were generated by PCR or gene synthesis and cloned using in-house mammalian expression vectors. The heavy and light chain sequences of the 645 Fab were cloned also separately using in-house mammalian expression vectors. All expression vectors were confirmed by direct sequencing using primers which covered the whole open reading frame.

Cultivating CHO cells: Suspensions of CHOS-XE cells (Cain, K., et al., Biotechnol Prog, 2013. 29(3): p. 697-706.) were pre-adapted in CD CHO medium (Invitrogen) supplemented with 2 mM Glutamax. Cells were kept in logarithmic growth phase with agitation at 120 RPM on a shaking incubator (Kuhner AG) and cultured at 37° C. in an atmosphere containing 8% CO₂.

Protein expression: The follistatin-Fab proteins were overexpressed by transient transfection of the CHO-XE cell line. Pairs of expression plasmids were co-transfected (e.g. N-fab light chain-FST-C with the heavy chain or FST-C fab heavy chain-C with the light chain). Immediately prior to transfection with DNA, CHO cells were exchanged in to Expi CHO expression medium (Gibco) by briefly centrifuging the cells at 1500×g and resuspending the pellet. Cells were then transfected using ExpiFectamine (Gibco), following manufacturer's instructions. The cultures were grown at 37° C. for the first 24 hours and then at 32° C. for the remainder of the expression cycle with shaking at 190 RPM in an atmosphere containing 8% CO₂. Supernatants were typically harvested 9-14 days post-transfection by centrifugation at 4000×g with subsequent filtration using 0.22 μm membranes. Final protein expression levels were determined by Protein G-HPLC and by SDS PAGE.

Protein purification: Transiently expressed protein content was captured using a Mab Select column (GE Healthcare) run under standard conditions. In short, the resin was washed with 10 column volumes of phosphate buffer saline (PBS, pH 7.4), and bound proteins were eluted with 5 column volumes of 0.1 M sodium citrate pH 3.1 (except if mentioned otherwise in the following examples). The eluate was neutralised with TRIS-HCl pH 8.5 and filter-sterilised through 0.22 μm membrane-exclusion chromatography (HiLoad 26/60 Superdex 75 column, GE Healthcare) run under standard conditions (here, columns were preloaded with PBS pH 7.4 as the running buffer). Sample quality was assessed using absorbance at 280 nm, BEH2000 analytical UPLC and SDS PAGE (under reducing and non-reducing conditions).

Adriamycin-induced kidney fibrosis model of chronic kidney disease (CKD): Female Balb/c mice, at least 6 weeks old and a weight greater than 24g, were subjected to an IV injection through the tail vein of cDNA (or pair of cDNA for paired studies) in an appropriate hydrodynamic transfection expression vector, one of which containing follistatin gene (Five Prime Therapeutics). After 7 days the mice were given adriamycin (doxorubicin) at 11 mg/kg IP and monitored for 49 days. Mice were monitored every day for weight and health; those mice that had lost greater than 40% of starting body weight or showing physical signs of end stage kidney failure were terminated and the data collected. At the end of the 49-day study period, all mice were terminated and kidneys and sera collected for analyses. Kidney hydroxyproline was measured using the QuickZyme assay method (QuickZyme Biosciences) and serum creatinine was measured using the 3-enzyme method and read out as pg/ml serum creatinine. Kidney fibrosis (hydroxyproline) in the saline plus adriamycin group, marked as “Saline” on the graphs, was considered to be a maximum disease response. This was set at 100% and used to normalize across the treatment groups. Naïve animals received no Adriamycin.

Statistical analysis methods specified for the in vivo analysis: Each assay run consisted of a Naïve group, a Saline Group and a number of cDNAs. In the primary screening stage, cDNAs were paired and, 15 mice per pair were treated; when retesting, cDNAs were tested singly with 22 mice per cDNA.

For each run, an analysis of variance (ANOVA) of log transformed data was performed for Hydroxyproline and for Serum Creatinine. An observation was deemed to be an outlier if its studentised residual was less than −3 or greater than +3. If outliers were detected, they were excluded and the analysis re-run. The cDNA group means were then tested vs. the Saline control group mean using a t-test based on the pooled residual standard deviation from the ANOVA. In the primary screen with n=15 per cDNA pair, a hit was declared if the difference vs. saline was significant at the 10% level (P=0.1). In the re-test runs, with n=22 per cDNA, a hit was declared if the difference vs. saline was significant at the 2.5% level (P=0.025). This strategy was adopted to produce acceptable false positive and false negative rates, while minimising the numbers of animals used.

PSR staining of mouse kidneys: A half kidney from each mouse was placed into 10% neutral buffered formalin, dehydrated in a tissue processor and paraffin embedded for histological analysis. Paraffin blocks were sectioned at 4 μm on a microtome and mounted on glass slides. Slides were then de-waxed and hydrated, stained with picrosirius red (PSR) before being washed with acidified water. Slides were then dehydrated, cleared and coverslipped. Slides were left to dry and then scanned using a Hamamatsu slide scanner. Whole slide images were imported into Definiens Developer XD 64 and analysed using the bright-field module. Briefly, cortex areas were manually annotated to remove the medulla from analysis. Marker threshold levels (e.g. PSR stain) were determined for the individual markers on a training data set of no fewer than four images. The analysis algorithm was then run on a dedicated server to determine marker areas above threshold intensity for each individual marker and the area of each marker above the threshold intensity was determined as a proportion of the area of the cortex.

Unilateral Ureteral Obstruction (UUO) induced kidney fibrosis model of chronic kidney disease: Male C57BL/6 mice (Charles River) weighing at least 19g were subjected to a Unilateral Ureteral Obstruction (UUO) induced kidney fibrosis model of chronic kidney disease. In those studies where the follistatin was delivered by hydrodynamic transfection, mice were transfected with 50 μg of either the pLIVE- human follistatin or pLIVE SEAP Vector (Mirus Bio, Cat #MIR 5320) in TranslT-EE vehicle. The solution containing the vector was injected rapidly (less than 2.5 seconds) intravenously in a volume of 2 ml. General anaesthesia with Isoflurane was induced prior to hydrodynamic injection and maintained for at least 1 minute post injection. In studies where mice were dosed with human follistatin protein, this was administered in 100 μl of saline via the subcutaneous route, at the dose concentrations specified in the Figure legends. Surgery was performed using Isoflurane induced general anaesthesia and under aseptic conditions as appropriate for surgical techniques. Mice underwent laparotomy followed by tying off the left ureter using 3.0 Mersilk ligature. The muscle wall was sealed using 5-0 Vicryl in a continuous pattern, with the skin being closed by subcuticular stitching using 5-0 Vicryl. Appropriate analgesia was administered pre-surgery and post-surgery. Blood samples were taken at appropriate times as detailed in the results. Mice were restrained using a recognised mouse restrainer and 30 μl of blood was taken from the tail vein via the tail prick method, using a pipette to accurately measure the blood sample. This sample was placed in a 0.2 ml PCR tube (Thermo) and spun down (20,000g, 5 minutes) to take off as much serum as possible, which was placed in a fresh PCR tube and stored at −80° C. On Day 19 or 21 mice were anaesthetised using Isoflurane, blood removed by cardiac puncture into serum tubes (Sarstedt) and the mice killed by cervical dislocation. The left kidney was removed and two quarters snap frozen in liquid nitrogen and stored at −80° C. The remaining half was placed into 10% neutral buffered formalin, dehydrated in a tissue processor and paraffin embedded for histological analysis and PSR staining as described above.

Human renal proximal tubule barrier integrity assay (see FIG. 5A for the principle): Primary human renal proximal tubule cells (Innoprot) were seeded onto 0.4 uM pore polycarbonate membranes in a 24 well HTS Transwell plate (Corning). Cells were cultured for 14 days, changing the medium (Advanced DMEM/F12 supplemented with renal epithelial growth kit from ATCC) every other day. Epithelial barrier function was disrupted by addition of 2 μM Adriamycin (also known as Doxorubicin—a well described nephrotoxic agent). To investigate the effects of Follistatin, some wells received Adriamycin plus either, 100 ng/ml Fab control protein or, 100 ng/ml FST315(HBM)-Fab. Following a 72 hr incubation, barrier integrity was assessed by adding 0.5 mg/ml FITC-Dextran (150 kDa) to the apical chamber and monitoring the appearance of the FITC signal in the basolateral chamber over time using a fluorescence plate reader. Percent permeability was calculated by dividing the fluorescence values from the test wells by the average signal detected in transwells without cells. The graph shows data points for individual wells with 6 replicates for each condition. Statistical significance was shown by Mann Whitney Student's T test.

### Example 1—Follistatin is Anti-Fibrotic in a Mouse Adriamycin-Induced Model of Chronic Kidney Disease (CKD)

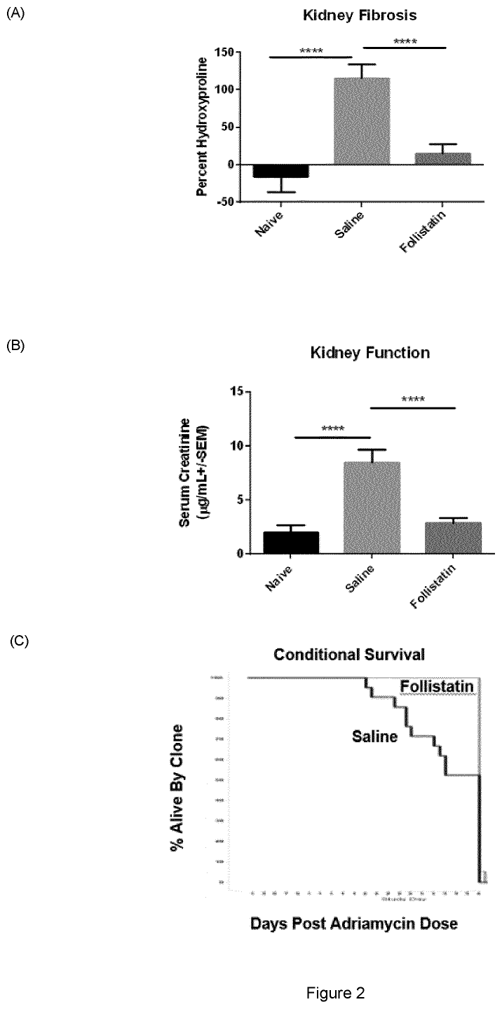

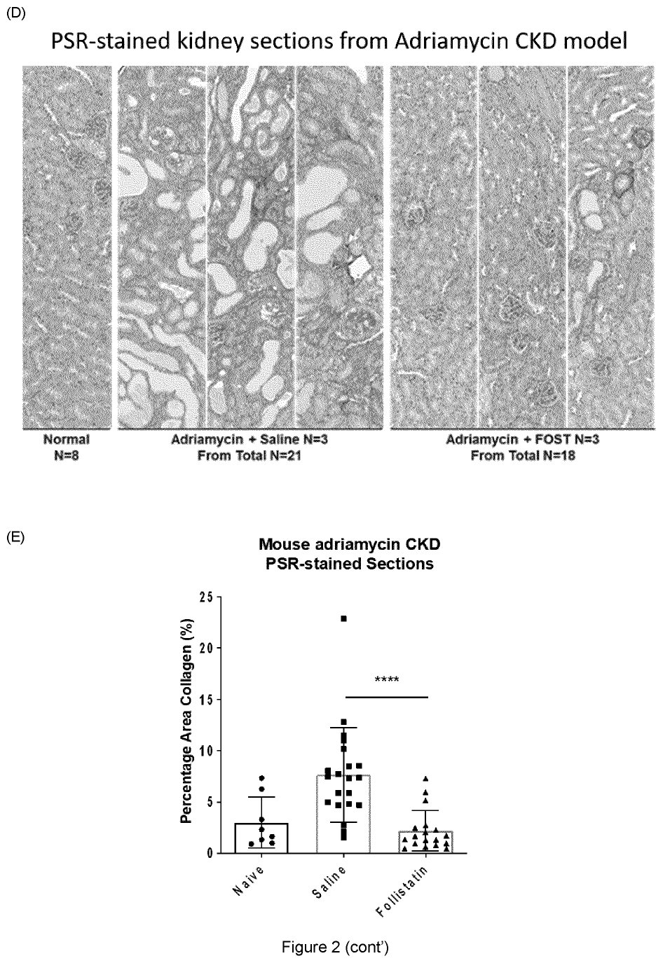

Mouse Follistatin was identified in a screen for fibrosis modifying proteins in the mouse Adriamycin CKD model initially as one member of a hydrodynamically transfected cDNA pair (n=15 mice). The anti-fibrotic effects of follistatin were confirmed in a repeat study, transfecting the follistatin cDNA independently in the same model (n=18-21 mice per group). Follistatin reduced kidney fibrosis as measured by total renal hydroxyproline 77% relative to the saline control group (p=0.0001) (FIG. 2A), while the loss of kidney function was returned to the normal range, as measured by serum creatinine (p=0.0001) (FIG. 2B) Conditional survival as measured by symptoms of end stage kidney failure increased from 50% in the saline treated Adriamycin group to 100% in the follistatin-treated group (FIG. 2C).

At 49 days, all kidneys from all groups ((naïve (n=8), untreated Adriamycin animals (n=21) and the follistatin hydrodynamically transfected animals (n=18)) were fixed, paraffin-embedded, sectioned and stained with either haemotoxylin and eosin (H&E) or picrosirius red (PSR) and imaged on a Hamamatsu slide scanner. Exemplar and representative images of the PSR-stained kidneys for one naïve, three untreated Adriamycin and three follistatin treated Adriamycin animals are shown in FIG. 2D. Untreated mice kidneys had extensive glomerulosclerosis and tubulointerstitial fibrosis with notable tubular basement membrane expansion, flattening of the tubular epithelium, extensive tubular dilation with loss of the brush border and tubular atrophy. In contrast the follistatin-treated mice were largely protected from both types of fibrotic remodelling. The 47 mouse kidney samples that were stained with PSR were subjected to high content image analysis using Definiens software and the area of PSR staining measured in the three groups was plotted as percentage PSR stained area (FIG. 2E). The follistatin-treated group PSR stained area was normalised (p<0.0001) relative to the 3-fold increase in untreated kidneys.

In conclusion, follistatin delivered by hydrodynamic transfection, profoundly inhibited multiple parameters of fibrosis and disease in the mouse adriamycin chronic kidney disease model including collagen accumulation, serum creatinine kidney damage biomarker levels, gross morphological kidney architecture and conditional survival of the mice for the duration of the study.

### Example 2—Follistatin Inhibits Renal Fibrosis in Mouse Unilateral Uretral Obstruction (UUO) Model of Acute Kidney Disease

The primary purpose of this study was to evaluate efficacy differences between different constructs of follistatin by hydrodynamic transfection (HDT) in delaying the onset of tubulointerstitial fibrosis in a UUO model.

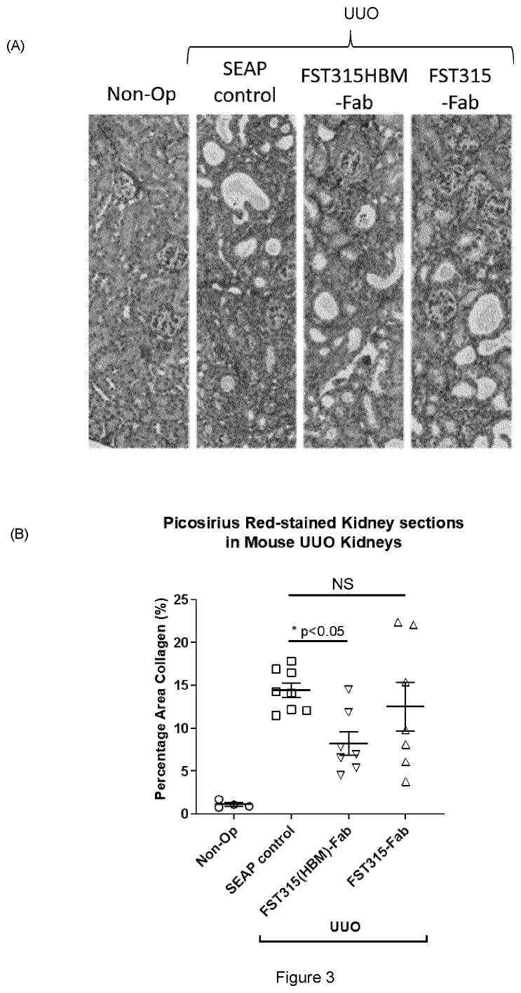

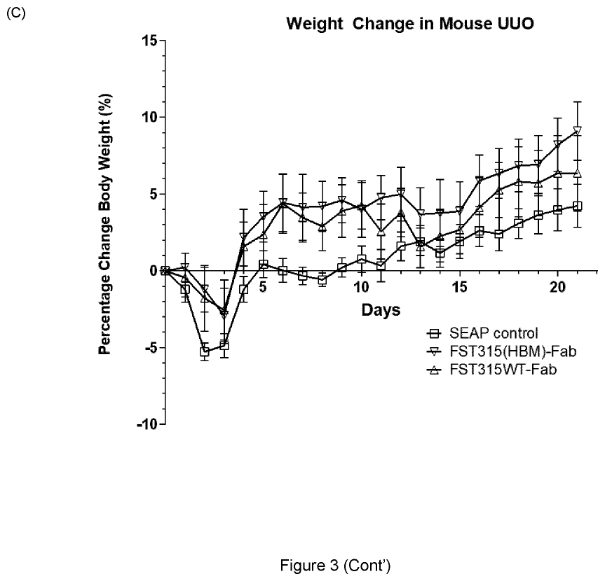

UUO kidneys in animals receiving the control SEAP vector by HDT demonstrated tubulointerstitial fibrosis in the cortex typical of this model at 21 days post-surgery with widespread expansion of the tubular basement membrane, strong collagen staining with the basement membrane particularly strongly stained within the medullary ray, widespread loss of epithelial cell volume, tubular atrophy, loss of proximal tubular brush border and extensive tubulointerstitial infiltrate. Less than 15% of tubules had near normal architecture. The medulla was effectively destroyed and missing while the changes to the glomeruli were minimal (FIG. 3A).

UUO kidneys in animals undergoing HDT with all follistatin moieties—FST315(HBM)-Fab and FST315-Fab, 1 day before UUO surgery showed reduced tubular basement expansion and collagen staining compared to UUO mice receiving the control SEAP vector. All follistatin-treated groups had preservation of tubular architecture with less epithelial flattening and atrophy occurring (FIG. 3A).

The picrosirius red (PSR) stained collagen sections were quantitated using definiens image analyses algorithms, showing the protective effect of follistatin. Although FST315-Fab showed a reduced percentage collagen area, this was not statistically significant; but 315Fst(HBM)-Fab (p<0.05) showed a significant inhibition of percentage collagen area relative to the SEAP control. This is in agreement with the visual histological assessment of the stained sections as described above (FIG. 3B).

Another indicator of follistatin-fusion protein efficacy is an increase of mouse body weight which was monitored daily for the duration of the study. Both FST315-Fab and FST315(HBM)-Fab showed less weight loss at the disease outset and gained more weight from day 4 onwards until termination of the study at day 21 (FIG. 3C).

In conclusion, a follistatin-fusion protein delivered by hydrodynamic transfection, was protective in a mouse acute kidney disease model, namely UUO, with disease parameters PSR staining and percentage collagen area of diseased kidneys and weight loss as the primary read-outs.

### Example 3—Comparison of FST315-Fab Versus FST315(HBM)-Fab, with a Dose Response Effect of the Latter, and Both Follistatin Moieties Delivered as Purified Proteins in a Mouse UUO Acute Kidney Disease Model

The primary purpose of this study was to compare the different FST315 protein formats for efficacy in delaying the onset of tubulointerstitial fibrosis in a UUO model.

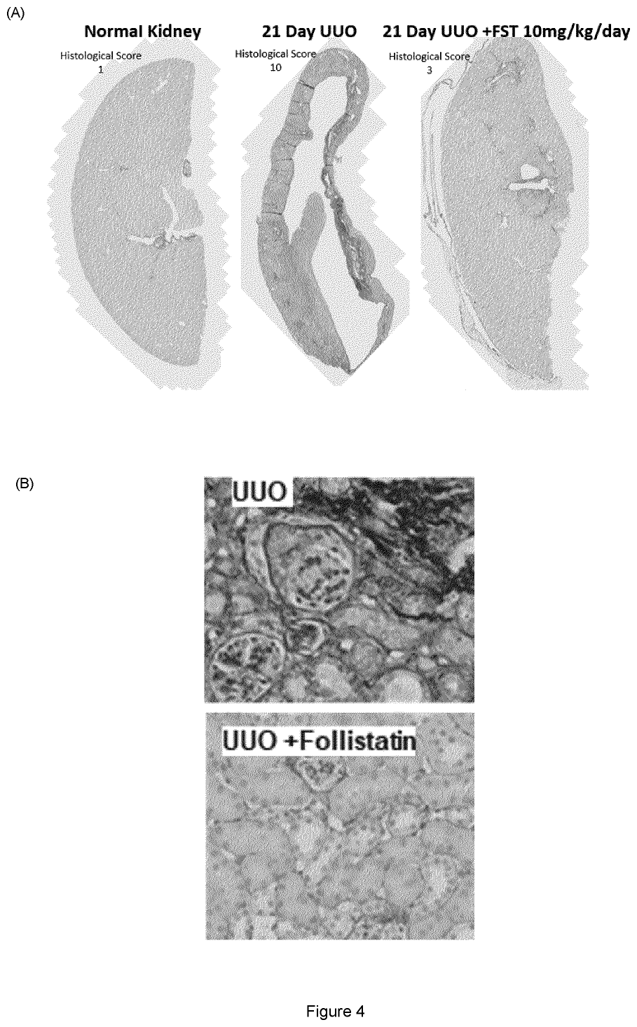

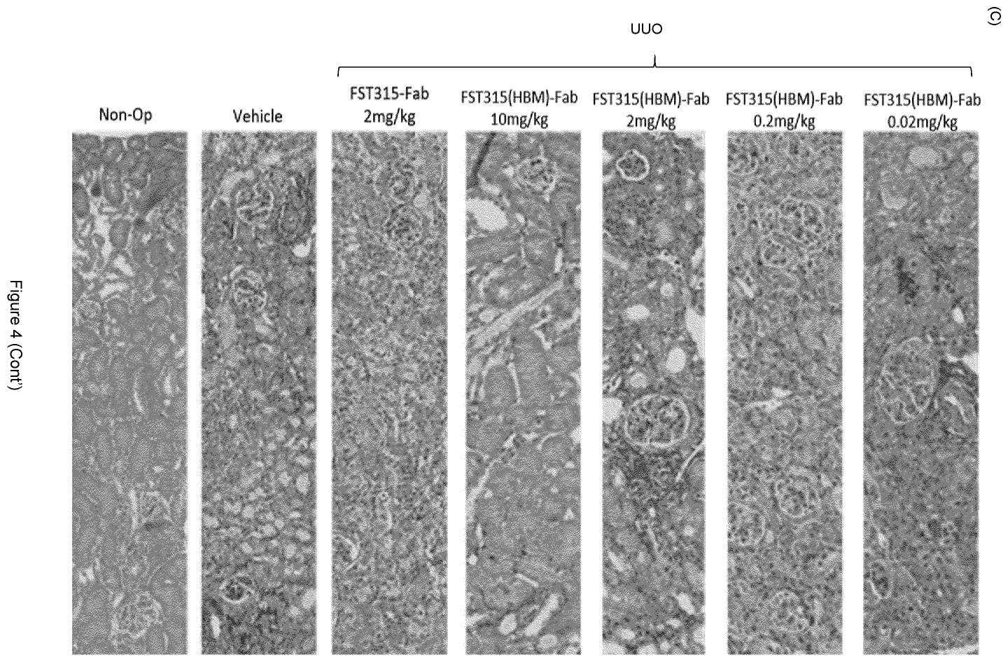

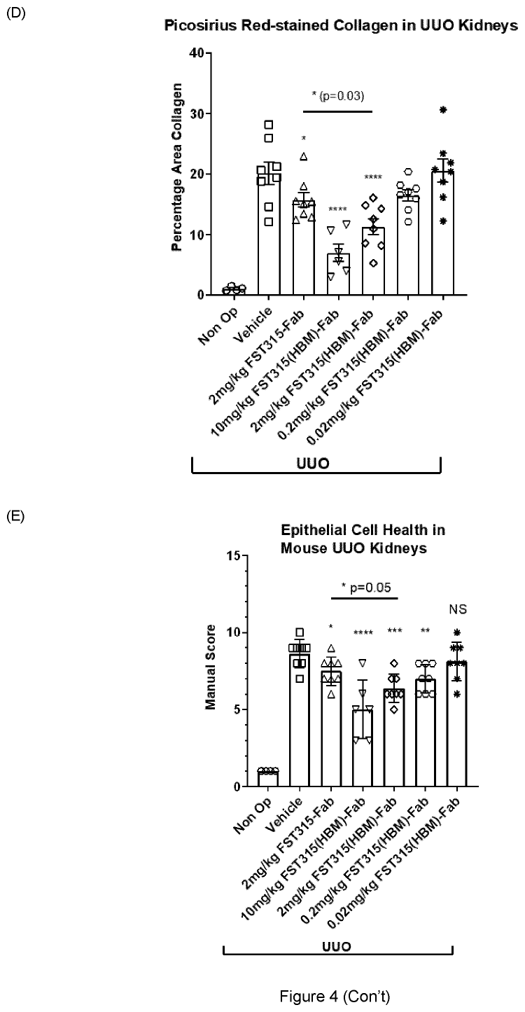

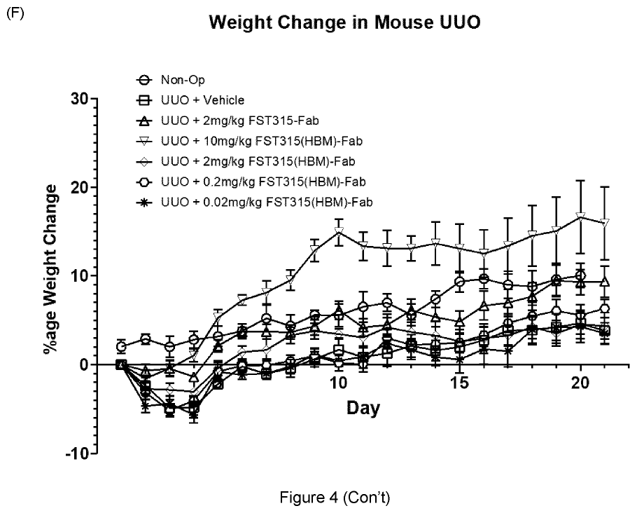

Examining the gross kidney morphology from PSR-stained kidneys in non-operated (non-diseased) versus 21-day UUO kidneys showed dramatic differences in shape, architecture and red staining and these gross changes could be reversed in the presence of FST315(HBM)-Fab at 10 mg/kg given every day; histological blind scoring quantitates these changes that are illustrated in FIG. 4A. A higher power magnification of the untreated 21-day UUO highlights the typical histological changes for this model and the normalization observed in the presence of high concentrations of FST315(HBM)-Fab (FIG. 4B).

FIG. 4C illustrates the effect of FST315-Fab versus FST315(HBM)-Fab in Day 21 UUO kidneys treated one day before surgery (Day−1) with the heparin-binding mutant version of the follistatin-Fab protein (FST315(HBM)-Fab) having a dramatically different appearance compared with vehicle-treated and also showing greater epithelial protection and less red collagen staining compared to wild-type follistatin-fab protein (FST315-Fab). There was reduced tubular atrophy and large sections of the cortex, especially in the 10 mg/kg dose, appeared almost normal with minimal basement membrane expansion, collagen staining or loss of architecture. Also, the FST315(HBM)-Fab effect on epithelial protection and collagen PSR staining was dose-dependent as there was a clear change from 10 mg/kg high dose with 2 mg/kg and 0.2 mg/kg intermediate doses showing less effects and 0.02 mg/kg dose barely distinguishable from untreated UUO kidney.

The picrosirius red (PSR) stained collagen sections were quantitated using definiens image analyses algorithms reading out on percentage collagen area, showing the dose-dependent protective significant effect of 315FST(HBM)-Fab fusion protein at 10 mg/kg (p<0.0001) and 2 mg/kg (p<0.001), with 0.2 and 0.02 mg/kg doses showing non-significant reductions (FIG. 4D); FST315-Fab at 2 mg/kg was statistically significantly different from the control untreated UUO group, p<0.05, even though the magnitude of the response was smaller. Importantly, FST315-Fab at 2 mg/kg was also statistically different from FST315(HBM)-Fab at 2 mg/kg (p=0.03), meaning that the protection afforded by the FST315(HBM)-Fab form was having additional effects mediated through the heparin-binding mutant changes. This could be due to better pharmacokinetic properties of FST315(HBM)-Fab which has a mean residence time in the mouse of 13.1 hours vs 9.3 hours for FST315-Fab or could be due to other functional characteristics that are not yet characterized. A histological manual scoring of the stained kidney sections in all the groups in the study was performed by an expert blinded to the groups, and the mean combined fibrotic scores were plotted in FIG. 4E. The same pattern was again observed in that the FST315(HBM)-Fab fusion protein had the most profound inhibitory effects with 10, 2 and 0.02 mg/kg showing significant differences, p<0.0001, p<0.001 and p<0.01, respectively from vehicle treated UUO kidneys. The FST315-Fab protein at 2 mg/kg did show significance in terms of epithelial protection versus vehicle-treated UUO at p<0.05 but this was inferior to the equivalent dose of the FST315(HBM)-Fab fusion format and also significantly different at p=0.05. This was in agreement with the visual histological assessment of the stained sections as described above.

All animals in the UUO study were weighed daily and the data plotted over time (FIG. 4F). The weight changes for the Follistatin moieties correlated closely with efficacy both for FST315(HBM)-Fab and FST315-Fab formats relative to the vehicle-treated UUO group; the different 315FST(HBM)-Fab doses also showed that weight gain is a good surrogate for kidney protection that can only be measured on study termination.

In conclusion, when administered as proteins FST315(HBM)-Fab showed statistically significant improvement over vehicle-treated disease animals and superior efficacy that was also statistically significant compared to FST315-Fab in protecting the mouse kidney from accrued damage initiated by UUO surgery, and this effect was dose-dependent. The superior protective effects on the kidney of FST315(HBM)-Fab were consistent across the morphological assessments, PSR-determined collagen content, epithelial cell health and gross body weight gains.

### Example 4—Effect of FST315(HBM)-Fab on Renal Proximal Tubule Epithelial Cell (RPTEC) Barrier Integrity

RPTEC cells grown in a 96-well transwell plate form a polarized, non-permeable barrier which can be measured by monitoring the resistance to diffusion of a fluorescent dye from the upper to the lower compartments of the transwell. In the presence of cytotoxic Adriamycin, the barrier integrity was compromised and could be measured by the presence of fluorescent dye, FITC-dextran (150 kDa) appearing in the basolateral supernatant. Treatment with FST315(HBM)-Fab, but not a Fab molecule alone, protected against this loss of barrier integrity which could be measured by partial restoration of permeability to FITC-dextran; this reduction in permeability with FST315(HBM)-Fab was statistically significant different from media control (p<0.0001). A diagram depicting the in vitro RPTEC model and the results are presented respectively FIGS. 5A and 5B). In conclusion, FST315(HBM)-Fab preserves primary RPTEC barrier integrity from Adriamycin-induced epithelial damage in an in vitro model of kidney barrier function.

### Example 5—FST-Fab and Fab-FST Exhibit Superior Protein Expression, Higher Monomeric Fraction Yield and Higher Half-Life than FST-WT or FST-Fc Fusion Proteins

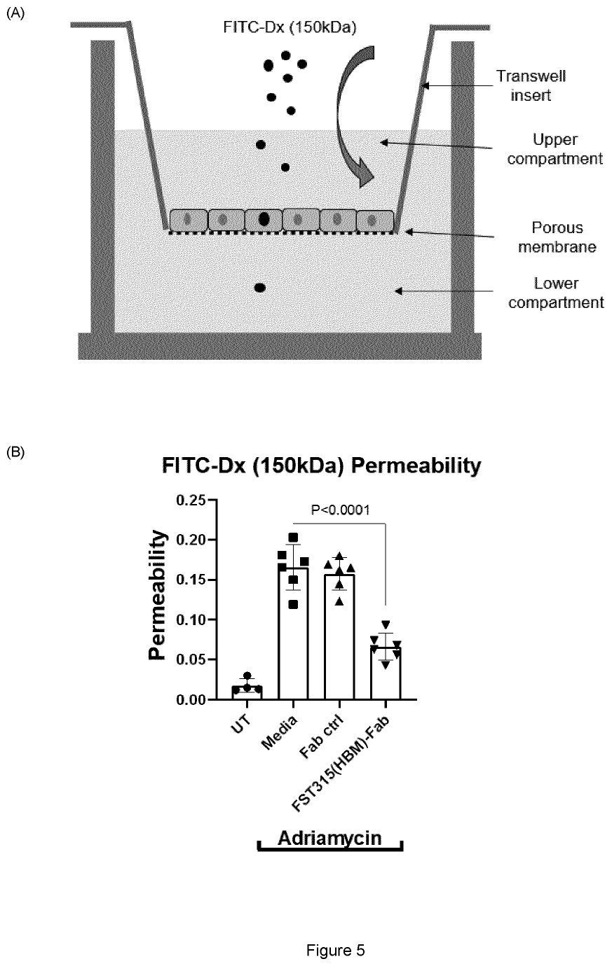

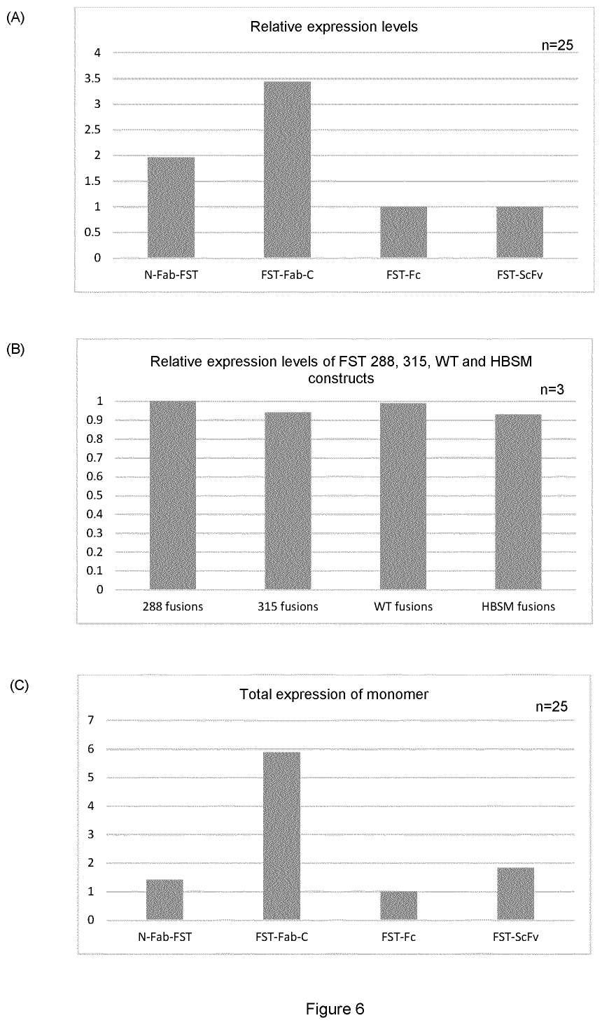

Comparing the relative expression levels of follistatin moiety fused with various fusion partners and in different orientations reveals the FST-Fab constructs fused in C-terminal of the FST moiety to be optimal (FIG. 6A). Having an Fc or a ScFv fused to the C-terminus of FST is significantly inferior to using a Fab at this position, both the former giving a 3.5-fold reduction in expressed product (FIG. 6A). Fusing a Fab moiety at the N-terminus of FST moiety leads to an about 45% drop in the amount of expressed product, although being better than fusion proteins comprising FST fused to an Fc domain. An assessment comparing the effect on expression using FST288- or FST315 fused to a Fab at the C-terminus of follistatin using wild-type of HBM sequences revealed only marginal differences (FIG. 6B). The FST315 version had expression 6% lower than the FST288 fusion. A comparison of fusions which contained the HBSM (HBM) revealed a modest 7% lowering in expression levels compared to wild-type.

A head-to-head examination of the various FST-fusions revealed Fab moieties fused to the C-terminus of the FST moiety to give the highest level of final monomer yield compared to the other fusions (FIG. 6C). The FST-Fc fusion gave the lowest yield of monomer amongst the set, being almost 6-fold worse than the FST-Fab fusion. The FST-ScFv and FST fused at the N-terminus of a Fab were approximately 3 and 4-fold worse, respectively.

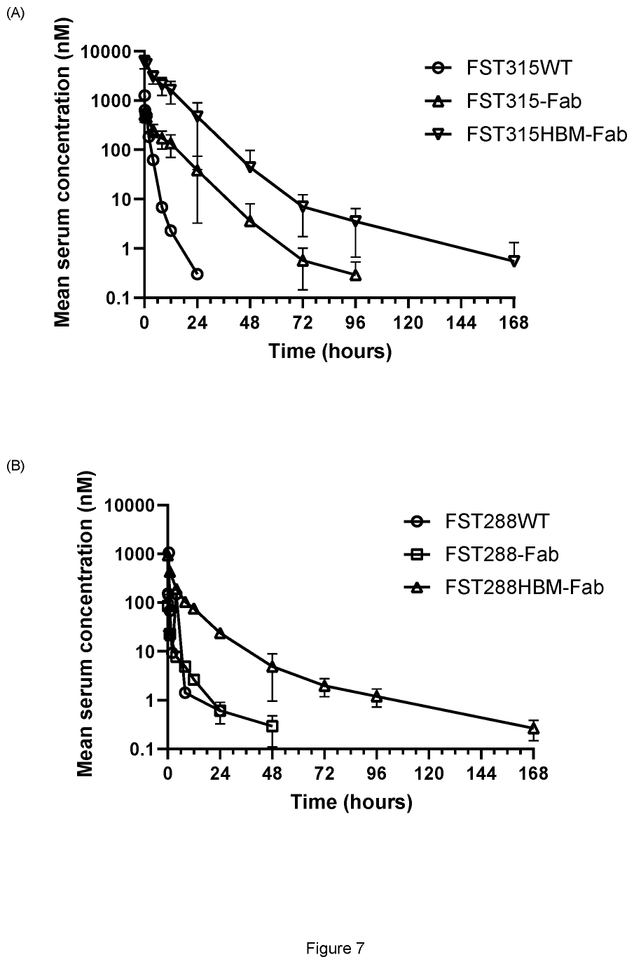

FST315WT, FST288WT and FST288-Fab administered intravenous (IV) at 10 mg/kg into mice were cleared very rapidly, with a mean residence time (MRT) of 1.6 hours, 2.3 hours and 5.2 hours, respectively. By comparison, FST315-Fab, FST315(HBM)-Fab and FST288HBM-Fab administered IV at 10 mg/kg into mice displayed extended kinetics, with an MRT of 9.3 hours, 13.1 hours and 11 hours, respectively, (FIGS. 7A and 7B). It is thus shown that it was possible to greatly extend the kinetics and half-life of a FST-containing protein thanks to the fusion between a FST moiety and a Fab moiety. A significant contribution to extended kinetics is also contributed by the mutation of the heparin binding site in the form of the HBM versions of the follistatin moieties.

### REFERENCES

1. WO2015/187977
2. WO2017/152090
3. WO03006057
4. Sidis et al. (2005) Endocrinology 146(1):130-136
5. http://blast.ncbi.nlm.nih.gov/
6. http://www.ebi.ac.uk/Tools/emboss/
7. WO2013/068571
8. Cain et al. (2013) Biotechnol Prog. 29(3): 697-706.
9. Sidis et al. (2006) Endocrinology 147(7):3586-3597
10. Yahr et al (2022) J. Osteopath. Med. 122 (1):55-63.
11. Ammirati A. L. (2020) Rev. Assoc. Med. Bras. 66: S3-S9.
12. Wynn & Ramalingam (2012) Nature Medicine 18 (7): 1028-1040.

1. (canceled)

2. A method of treating a patient in need of therapy for the treatment, amelioration or prevention of kidney diseases or kidney damage, the method comprising administering a therapeutically effective amount of a follistatin-fusion protein, or of a pharmaceutical composition comprising a follistatin-fusion protein, wherein the follistatin-fusion protein comprises:
a. a follistatin moiety,
b. an antibody moiety, and optionally
c. a linker between the follistatin moiety and the antibody moiety.

3. (canceled)

4. The method according to claim 2, wherein the kidney disease is a chronic kidney disease such as primary glomerulonephritis, secondary glomerulonephritis, diabetic nephropathy, hypertensive nephrosclerosis, focal segmental glomerulosclerosis (FSGS), IgA nephropathy (IgAN), Mesangial proliferative glomerulonephritis, Membranous Nephropathy (MN), Minimal Change Disease (MCD), polycystic kidney disease, chronic allograft nephropathy, CKD-associated mineral and bone disorder (CKD-MBD) and Goodpastures disease.

5. The method according to claim 2, wherein the kidney damage is a) kidney fibrosis, or b) is associated with other diseases such as diabetes mellitus, hypertension, cardiovascular disease or bone disorder.

6. The the method according to claim 2, wherein the kidney disease or kidney damage is the result of a therapeutic treatment.

7. The method according to claim 6, wherein the kidney disease or kidney damage is the result of treatment with chemotherapy.

8. The method according to claim 7, wherein the chemotherapy is selected from Ara-C, bleomycin, camptothecin, carboplatin, cisplatin, cyclophosphamide, doxorubicin (Adriamycin), gemcitabine, methotrexate, paclitaxel and vincristine.

9. The method according to claim 7, wherein the treatment or amelioration of kidney disease or kidney damage comprises administering the fusion protein simultaneously, separately or sequentially with the chemotherapy.

10. The method according to claim 2, wherein the follistatin moiety comprises or is a naturally occurring protein or a functional variant thereof.

11. The method according to claim 2, wherein the follistatin moiety is selected from:
a. SEQ ID NO: 1.
b. SEQ ID NO: 2
c. SEQ ID NO: 3
d. SEQ ID NO: 4
e. any protein comprising amino acid residues comprising between 289 and 314 residues of any one of SEQ ID Nos.1 to 4; or
f. a sequence having at least 95%, at least 96%, at least 97%, at least 98% or at least 99% sequence identity to any one of SEQ ID NOs: 1 to 4.

12. The method according to claim 2, wherein the antibody moiety binds albumin, and optionally wherein it binds human serum albumin (HSA).

13. The method according to claim 2, wherein the antibody moiety is a chimeric, humanized or human antibody moiety.

14. The method according to claim 2, wherein the antibody moiety is an antigen-binding fragment selected from a Fab, a Fab′ or a F(ab′)2.

15. The method according to claim 2, wherein the antibody moiety comprises:
a. a light chain variable region comprising a CDR-L1 comprising SEQ ID NO: 13; a CDR-L2 comprising SEQ ID NO: 14 and a CDR-L3 comprising SEQ ID NO: 15; and a heavy chain variable region comprising a CDR-H1 comprising SEQ ID NO: 16; a CDR-H2 comprising SEQ ID NO: 17 and a CDR-H3 comprising SEQ ID NO: 18; or
b. a heavy chain variable region comprising or consisting of SEQ ID NO: 5 and a light chain variable region comprising or consisting of SEQ ID NO: 6.

16. The method according to claim 2, wherein the linker is selected from the group consisting of: SGGGGS (SEQ ID NO: 7), SGGGGSSGGGGS (SEQ ID NO: 19), GGGGS (SEQ ID NO: 20) and GGGGSGGGGS (SEQ ID NO: 21).

17. The method according to claim 2, wherein a) the antibody moiety is connected to the C-terminal or N-terminal portion of the follistatin moiety or, b) if a linker is present, the antibody moiety is connected to the C-terminal portion of the follistatin moiety by the linker.

18. The method according to claim 2, wherein the fusion protein comprises or consists of:
(a) i. an FST315 polypeptide defined by SEQ ID NO: 1, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide; and
iii. a Fab light chain defined by SEQ ID NO: 6, or a sequence having at least 95%, at least 96%, at least 97%, at least 98% or at least 99% sequence identity thereto;
(b) i. an FST288 polypeptide defined by SEQ ID NO: 2, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide; and
iii. a Fab light chain defined by SEQ ID NO: 6, or a sequence having at least 95%, at least 96%, at least 97%, at least 98% or at least 99% sequence identity thereto;
(c) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST315 polypeptide variant; and
iii. a Fab light chain defined by SEQ ID NO: 6, or a sequence having at least 95%, at least 96%, at least 97%, at least 98% or at least 99% sequence identity thereto;
(d) i. an FST288 polypeptide variant defined by SEQ ID NO: 4 (FST288HBM) or SEQ ID NO: 25, or a sequence having at least 95%, at least 96%, at least 97%, at least 98% or at least 99% sequence identity thereto;
ii. a Fab heavy chain defined by SEQ ID NO: 5, or a sequence having at least 95%, at least 96%, at least 97%, at least 98% or at least 99% sequence identity thereto, conjugated to the C-terminus of the FST288 polypeptide variant; and
iii. a Fab light chain defined by SEQ ID NO: 6, or a sequence having at least 95%, at least 96%, at least 97%, at least 98% or at least 99% sequence identity thereto;
(e) i. an FST315 polypeptide defined by SEQ ID NO: 1, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(f) i. an FST288 polypeptide defined by SEQ ID NO: 2, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(g) i. an FST315 polypeptide variant defined by SEQ ID NO: 3 (FST315HBM) or SEQ ID NO: 22, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST315 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(h) i. an FST288 polypeptide variant defined by SEQ ID NO: 4 (FST288HBM) or SEQ ID NO:25, or a sequence having at least 95%, at least 96%, at least 97%, at least 98% or at least 99% sequence identity thereto;
ii. a linker defined by SEQ ID NO: 7, SEQ ID NO: 19, SEQ ID NO: 20 or SEQ ID NO: 21 conjugated to the C-terminus of the FST288 polypeptide variant;
iii. a Fab heavy chain defined by SEQ ID NO: 5, or a sequence having at least 95%, at least 96%, at least 97%, at least 98% or at least 99% sequence identity thereto conjugated to the free-terminus of the linker; and
iv. a Fab light chain defined by SEQ ID NO: 6, or a sequence having at least 95%, at least 96%, at least 97%, at least 98% or at least 99% sequence identity thereto;
(i) (i) SEQ ID NO: 8, 9, 10, 11, 24, 25, 26, 27, 32, 33, 34 or 35, or a sequence having at least 95%, at least 96%, at least 97%, at least 98% or at least 99% sequence identity thereto, together with a Fab light chain defined by SEQ ID NO: 6 or a sequence having at least 95%, at least 96%, at least 97%, at least 98% or at least 99% sequence identity thereto;
(j) SEQ ID NO: 8, 9, 10, 11, 24, 25, 26, 27, 32, 33, 34 or 35, together with a Fab light chain defined by SEQ ID NO: 6; or
(k) a functional variant or fragment of any one of (a) to (j).


SEQUENCE LISTING (35 sequences)

SEQ ID NO: 1 - protein, 315 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 2 - protein, 288 aa, Homo sapiens
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 3 - protein, 315 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 4 - protein, 288 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 5 - protein, 224 aa, synthetic construct
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 6 - protein, 217 aa, synthetic construct
DIQMTQSPSSVSASVGDRVTITCQSSPSVWSNFLSWYQQKPGKAPKLLIYEASKLTSGVPSRFSGSGSGTDFTLTISSLQPEDFATYYCGGGYSSISDTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 6 aa, synthetic construct
SGGGGS

SEQ ID NO: 8 - protein, 545 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 9 - protein, 539 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 10 - protein, 518 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 11 - protein, 512 aa, synthetic construct
GNCWLRQAKNGRCQVLYKTELSKEECCSTGRLSTSWTEEDVNDNTLFKWMIFNGGAPNCIPCKETCENVDCGPGKACRMNAANKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 12 - protein, 29 aa, synthetic construct
MVRARHQPGGLCLLLLLLCQFMEDRSAQA

SEQ ID NO: 13 - protein, 12 aa, synthetic construct
QSSPSVWSNFLS

SEQ ID NO: 14 - protein, 7 aa, synthetic construct
EASKLTS

SEQ ID NO: 15 - protein, 11 aa, synthetic construct
GGGYSSISDTT

SEQ ID NO: 16 - protein, 10 aa, synthetic construct
GIDLSNYAIN

SEQ ID NO: 17 - protein, 16 aa, synthetic construct
IIWASGTTFYATWAKG

SEQ ID NO: 18 - protein, 13 aa, synthetic construct
TVPGYSTAPYFDL

SEQ ID NO: 19 - protein, 12 aa, synthetic construct
SGGGGSSGGGGS

SEQ ID NO: 20 - protein, 5 aa, synthetic construct
GGGGS

SEQ ID NO: 21 - protein, 10 aa, synthetic construct
GGGGSGGGGS

SEQ ID NO: 22 - protein, 315 aa, synthetic construct
  VARIANT 76: note = X = any amino acid
  VARIANT 81..82: note = X = any amino acid
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 23 - protein, 288 aa, synthetic construct
  VARIANT 76: note = X = any amino acid
  VARIANT 81..82: note = X = any amino acid
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 24 - protein, 539 aa, synthetic construct
  VARIANT 76: note = X = any amino acid
  VARIANT 81..82: note = X = any amino acid
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 25 - protein, 545 aa, synthetic construct
  VARIANT 76: note = X = any amino acid
  VARIANT 81..82: note = X = any amino acid
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 26 - protein, 512 aa, synthetic construct
  VARIANT 76: note = X = any amino acid
  VARIANT 81..82: note = X = any amino acid
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 518 aa, synthetic construct
  VARIANT 76: note = X = any amino acid
  VARIANT 81..82: note = X = any amino acid
GNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 539 aa, synthetic construct
  VARIANT 300: note = X = any amino acid
  VARIANT 305..306: note = X = any amino acid
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 29 - protein, 545 aa, synthetic construct
  VARIANT 306: note = X = any amino acid
  VARIANT 311..312: note = X = any amino acid
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 30 - protein, 512 aa, synthetic construct
  VARIANT 300: note = X = any amino acid
  VARIANT 305..306: note = X = any amino acid
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 31 - protein, 518 aa, synthetic construct
  VARIANT 306: note = X = any amino acid
  VARIANT 311..312: note = X = any amino acid
EVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCSGGGGSGNCWLRQAKNGRCQVLYKTELSKEECCSTGRLSTSWTEEDVNDNTLFKWMIFNGGAPNCIPCKETCENVDCGPGKXCRMNXXNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 32 - protein, 545 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 33 - protein, 539 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEWEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 518 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSGGGGSEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 35 - protein, 512 aa, synthetic construct
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNEVQLLESGGGLVQPGGSLRLSCAVSGIDLSNYAINWVRQAPGKGLEWIGIIWASGTTFYATWAKGRFTISRDNSKNTVYLQMNSLRAEDTAVYYCARTVPGYSTAPYFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC